(19) United States
(12) Patent Application Publication
(10) Pub. No.: US 2002/0155571 A1
(43) Pub. Date: Oct. 24, 2002

(54) Triazine degrading enzymes

(76) Inventors: Sun Ai Raillard, Mountain View, CA (US); Jeremy Minshull, Minshull, CA (US); Claes Gustafsson, Belmont, CA (US)

Correspondence Address: LAW OFFICES OF JONATHAN ALAN QUINE, P O BOX 458, ALAMEDA, CA, 94501

(21) Appl. No.: 09/796,990

(22) Filed: Feb. 28, 2001

Related U.S. Application Data
(60) Provisional application No. 60/185,809, filed on Feb. 29, 2000.

(51) Int. Cl. C12P 17/10; C12N 9/16; C07H 21/04; C12P 21/02; C12N 5/06

(52) U.S. Cl. 435/196; 435/320.1; 435/69.1; 435/325; 536/23.2; 435/121

(57) ABSTRACT

New triazine hydrolases, (both nucleic acids and proteins) are provided. Compositions which include these new proteins and/or genes, recombinant cells, shuffling methods involving the new triazine hydrolases, antibodies to the new triazine hydrolases and methods of using the triazine hydrolases are also provided.

### CROSS-REFERENCE TO RELATED APPLICATIONS

Pursuant to 35 U.S.C. § 119(e) and any other applicable statute or rule, the present application claims benefit of and priority to U.S. Ser. No. 60/185,809 “Triazine Degrading Enzymes,” by Bermudez et al., filed Feb. 29, 2000, and co-filed PCT application, “Triazine Degrading Enzymes,” by Bermudez et al., filed Feb. 27, 2001, Attorney Docket No. 02-104510PC.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Atrazine and other triazine derivatives are widely used as herbicides for broad leaf weed control. Approximately 800 million pounds of these compounds were used in the U.S. between 1980 and 1990. As a result of this widespread use, the compounds have been detected in ground and surface water in the U.S. and in many other countries that use triazine derivatives.

Atrazine and related compounds are degraded very slowly in nature. For example, atrazine has a water solubility of 33 mg/liter at 27° C. and its half-life can vary from about 4 to about 60 weeks when present in soil. Therefore, high concentrations of triazine derivatives in soil can take quite a long time to dissipate.

Isolation of triazine and/or atrazine degrading microorganisms has been reported by numerous sources. See, e.g., Behki et al., J. Agric. Food Chem. 34, 746-749 (1986); Behki et al., Appl. Environ. Microbiol. 59, 1955-1959 (1993); Cook, FEMS Microbiol. Rev. 46, 93-116 (1987); Cook et al., J. Agric. Food Chem. 29, 1135-1143 (1981); Erikson et al., Critical Rev. Environ. Cont. 19, 1-13 (1989); Giardina et al., Agric. Biol. Chem. 44, 2067-2072 (1980); Jesse et al;, Appl. Environ. Microbiol. 45 97-102 (1983); Mandelbaum et al., Appl. Environ. Microbiol. 61, 1451-1457 (1995); Mandelbaum et al., Appl. Environ. Microbiol. 59, 1696-1701 (1993); Mandelbaum et al., Environ. Sci. Technol. 27, 1943-1946 (1993); Radosevich et al., Appl. Environ. Microbiol. 61, 297-302 (1995); and Yanze-Kontchou et al., Appl. Environ. Microbiol. 60 4297-4302 (1994).

Genes encoding atrazine degrading enzymes, e.g., atrazine chlorohydrolases, have also been isolated. See, e.g., Souza et al., Appl. Environ. Microbiol. 61, 3373-3378 (1995). While this protein is useful for dechlorinating atrazine, improved triazine hydrolases are desirable.

The present invention provides novel triazine hydrolases that provide novel enzyme substrate activity. These hydrolases are useful in a variety of soil and water treatments and other industrial and commercial applications that will be apparent upon further review.

### SUMMARY OF THE INVENTION

The present invention provides novel triazine hydrolases with improved characteristics such as activity against a wider range of substrates than wild type triazine hydrolases. In one aspect, the invention provides isolated and recombinant nucleic acids corresponding to polynucleotides that are novel triazine hydrolases, encode novel triazine hydrolase proteins, hybridize under highly stringent conditions to such novel triazine hydrolases or polynucleotides encoding novel triazine hydrolase proteins, or fragments thereof encoding polypeptides with triazine hydrolase activity.

In one embodiment, the invention provides polynucleotides which include a subsequence corresponding to one or more of SEQ ID NO: 1 to SEQ ID NO: 48 or a complementary polynucleotide sequence thereof. Polynucleotide sequences encoding a polypeptide selected from SEQ ID NO: 49 to SEQ ID NO: 608, or a complementary polynucleotide sequence thereof are also provided. Polynucleotide sequences which hybridize under highly stringent conditions over substantially the entire length of one or more of the above polynucleotide sequences provide additional embodiments. Other embodiments include fragments of the above sequences, which fragments typically have triazine hydrolase activity.

In preferred embodiments, the nucleic acids of the invention comprise a polynucleotide that encodes a hydrolase, e.g., a triazine hydrolase. The triazine hydrolase typically hydrolyzes one or more of: aminoatrazine, atrazine, triazine, atratone, N-methylatrazine, ametryn, aminopropazine, propazine, prometon, N-methylpropazine, prometryn, aminomorphazine, morphazine, morphatryn, morphaton, or N-methylmorphazine. The encoded polypeptide is typically about 450 to about 500 amino acids in length or about 474 amino acids in length. Polypeptides comprising at least about 20 contiguous amino acids, at least about 50 contiguous amino acids, at least about 100 contiguous amino acids, or at least 150 contiguous amino acids of any one of SEQ ID NO: 49-608 are also provided.

In another aspect, the invention provides a cell comprising any of the nucleic acids described above. Such cells typically express a polypeptide encoded by one of the nucleic acids of the invention.

In another aspect, vectors comprising the nucleic acids of the invention are provided. The vector, e.g., an expression vector, typically comprises a plasmid, a cosmid, a phage, a virus, or the like. Cells transduced by such vectors are also provided.

In another aspect, the invention provides remediation compositions comprising a cell comprising the polypeptides or polynucleotides of the invention. The remediation compositions are typically used to treat or decontaminate triazine or atrazine contaminated water, soil, or the like. Such remediation compositions are optionally used in the methods of the invention. For example, a method of treating a sample comprising atrazine or a triazine derivative is provided. The method comprises adding a composition to a sample comprising atrazine or a triazine derivative, wherein the composition comprises a polypeptide encoded by a nucleic acid of the invention. The nucleic acids and polypeptides of the invention are thus used to decontaminate triazine contaminated soil, water, or the like.

Compositions containing two or more nucleic acids of the invention are an additional feature of the invention. In some cases, these compositions are libraries of nucleic acids, preferably containing at least ten such nucleic acids. Compositions produced by digesting one or more nucleic acids of the invention, e.g., with a restriction endonuclease, an RNAse, or a DNAse, are also a feature of the invention, as are compositions produced by incubating one or more nucleic acids of the invention, e.g., in the presence of deoxyribonucleotide triphosphates and a nucleic acid polymerase, such as a thermostable polymerase.

Isolated or recombinant polypeptides encoded by the nucleic acids of the invention are also provided. For example, polypeptides comprising a sequence selected from SEQ ID NO: 49-608 are provided. These polypeptides typically have triazine hydrolase activity of at least 50,000 nM per hour or about 2-fold to at least about 200-fold greater than an atrazine chlorohydrolase corresponding to U55933. Polypeptides comprising about 100 contiguous amino acids, at least about 150 contiguous amino acids of the encoded protein, or at least about 250 contiguous amino acids of the encoded protein are also provided.

Furthermore, polypeptides of the invention with secretion/localization sequences are a feature of the invention, as are polypeptides with purification subsequences, including epitope tags, FLAG tags, polyhistidine tags, GST fusions, and the like. Similarly, polypeptides of the invention bearing a methionine at the N-terminus or comprising one or more modified amino acid, e.g., a glycosylated amino acid, a PEGylated amino acid, a farnesylated amino acid, an acetylated amino acid, a biotinylated amino acid, or the like, are features of the invention.

Polypeptides that are specifically bound by a polyclonal antisera raised against one or more antigen derived from SEQ ID NO: 49-608, or a fragment thereof, wherein the antisera is subtracted with a naturally occurring hydrolase polypeptide corresponding to U55933 or a triazine hydrolase homologue nucleic acid that is present in a public database such as GenBank™ at the time of filing of the subject application as well as antibodies or antisera produced by administering such polypeptides to a mammal and antibodies or antisera that specifically bind a polypeptide of the invention and do not specifically bind to naturally-occurring or recombinant hydrolase polypeptides corresponding to U55933 are all features of the invention.

In another aspect, the invention provides methods of producing polypeptides. The methods typically comprise introducing into a population of cells a nucleic acid or recombinant expression vector of the invention. The nucleic acid is generally operatively linked to a regulatory sequence effective to produce the encoded polypeptide. The cells are cultured in a culture medium to produce the polypeptide, which is isolated from the cells or from the culture medium.

Another aspect of the invention relates to DNA shuffling to provide novel triazine hydrolase homologues by recursively recombining one or more nucleic acid of the invention with one or more additional nucleic acid, such as a nucleic acid encoding a triazine hydrolase homologue or subsequence thereof. In one embodiment, recursive recombination produces at least one library of recombinant triazine hydrolase homologue nucleic acids. The libraries so produced are embodiments of the invention as are cells comprising the libraries. Furthermore, methods of producing modified triazine hydrolases by mutating the nucleic acids of the invention are provided. Recombinant and mutant triazine hydrolase nucleic acids produced by the methods of the invention are also embodiments of the invention.

In addition, nucleic acids comprising unique subsequences selected from SEQ ID NO: 1 to SEQ ID NO: 48, (as compared to a nucleic acid corresponding to U55933); polypeptides comprising a unique subsequence from: SEQ ID NO: 49-608, (as compared to a polypeptide corresponding to U55933); and target nucleic acids that hybridize under stringent conditions to a unique coding oligonucleotide that encodes a unique subsequence of a polypeptide selected from: SEQ ID NO: 49-608, (unique as compared to a polypeptide corresponding to U55933) are also features of the invention.

The invention also provides computers, computer readable mediums and integrated systems, including databases that are composed of sequence records including character strings corresponding to SEQ ID NO: 1 to SEQ ID NO: 608. Such integrated systems optionally include one or more instruction set for selecting, aligning, translating, reverse-translating, or viewing any of the above character strings with each other and/or with any additional nucleic acid or amino acid sequence.

### BRIEF DESCRIPTION OF THE FIGURES

FIG. 1 illustrates the conversion of atrazine to hydroxyatrazine via dechlorination catalyzed by atrazine chlorohydrolase.

FIG. 2 defines and illustrates a variety of triazine derivatives designed to explore chemical space. One side chain is fixed as isopropyl amine and the second side chain (R1) and the leaving group (R2) are varied, e.g., to confer increased bulkiness.

FIG. 3 provides turnover rates in nM substrate/h/20 μl cells (A600=3.0) for atrazine chlorohydralase and a preferred library member with activity towards each of ten substrates with either ethyl or isopropyl amine R1 groups.

FIG. 4 provides a distribution of functional activity in sequence space. Triazine hydrolase activities towards each of 15 substrates is indicated by a circle whose area is proportional to the activity. Substrates are arranged in a grid format as in FIG. 2. Activities are shown on a phylogenetic tree to show relationships between enzyme sequences.

### DETAILED DISCUSSION OF THE INVENTION

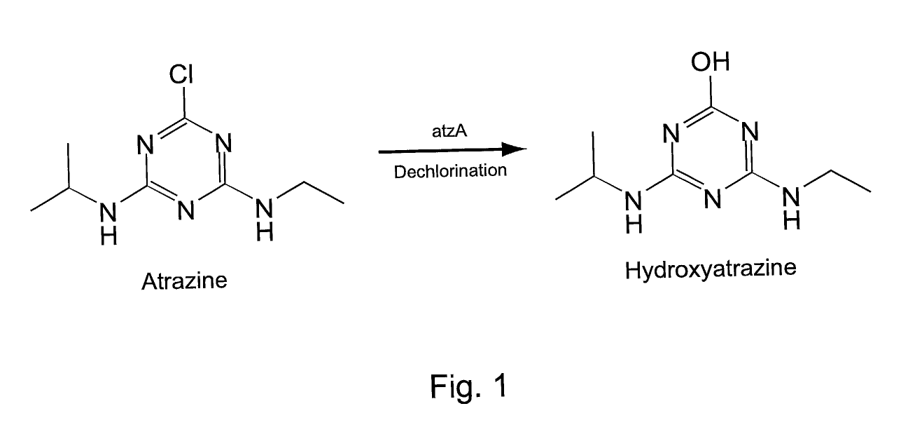

Atrazine and other triazine derivatives are widely used in herbicides, either alone or in combination with other compounds, e.g., for control of broad-leaf weeds. Atrazine and triazine runoff and persistence in soil and water often lead to triazine levels exceeding EPA limits for drinking water. Solutions to these concerns, e.g., atrazine contaminated soil, involve introduction of indigenous and/or recombinant bacteria to metabolize or degrade triazine compounds in soil or water. Wild-type hydrolases present in indigenous bacteria degrade atrazine at low levels but do not degrade or metabolize other triazine derivatives. FIG. 1 illustrates the conversion of atrazine to hydroxyatrazine using atrazine chlorohydrolase.

The present invention provides novel triazine hydrolases with triazine degradation activity, e.g., hydrolysis of atrazine as well as degradation activity with respect to other triazine derivatives that are not metabolized by wild type hydrolases. Triazine hydrolase refers to an enzyme or polypeptide having triazine hydrolase activity, i.e., the ability to hydrolyze or otherwise degrade one or more species of the class of compounds represented by:

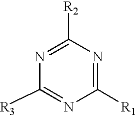

wherein R₁ and R₃ each independently comprise an amino group, i.e., —NH₂, or a substituted linear, branched, or cyclic amino group. Typically R₁ and R₃ are each independently a lower-alkyl-substituted amino group or a morpholino group. As used herein, the term “lower alkyl” refers to a C₁₋₆ alkyl. More typically, R₁ and R₃ are each independently —NH(C₂H₅), —NHCH(CH₃)₂,

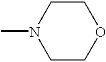

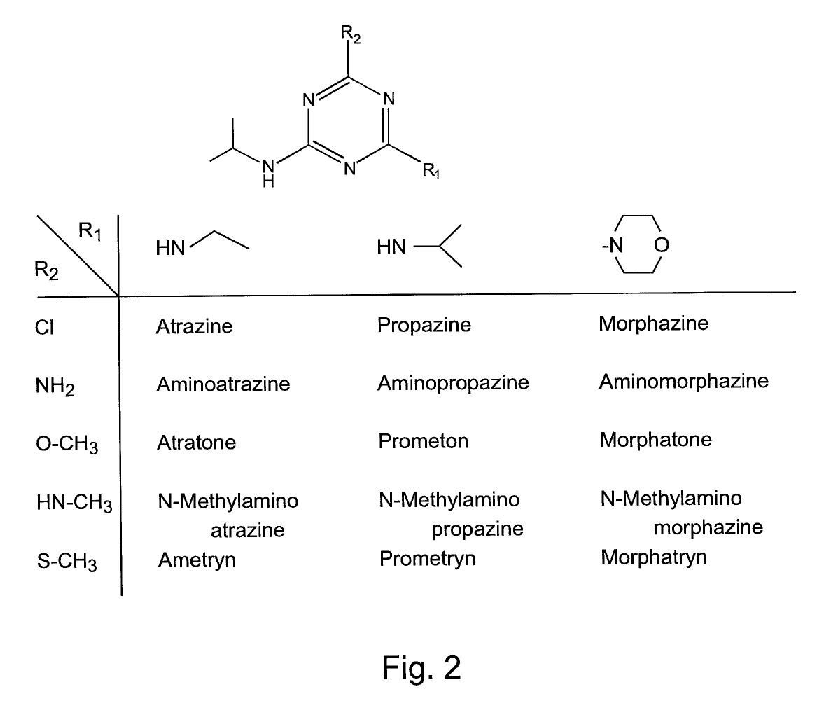

or the like. Preferably, R₃ is —NHCH(CH₃)₂, R₂ is an amino group, i.e., —NH₂, or an optionally substituted amino group, e.g., —NRH or —NRR′, a halo, a lower alkoxy, or —S—R, where R and R′ are each independently a lower alkyl group. Typically R₂ is —NH₂, —X, wherein X is a halogen such as Cl, —OCH₃, —NH(CH₃), or —S—CH₃. Example compounds are shown and defined by FIG. 2, which defines the R groups for a variety of triazine compounds.

Definitions

A “polynucleotide sequence” is a nucleic acid (which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

Similarly, an “amino acid sequence” is a polymer of amino acids (a protein, polypeptide, etc.) or a character string representing an amino acid polymer, depending on context. Either the given nucleic acid or the complementary nucleic acid can be determined from any specified polynucleotide sequence.

A nucleic acid, protein or other component is “isolated” when it is partially or completely separated from components with which it is normally associated (other proteins, nucleic acids, cells, synthetic reagents, etc.). A nucleic acid or polypeptide is “recombinant” when it is artificial or engineered, or derived from an artificial or engineered protein or nucleic acid.

A “subsequence” or “fragment” is any portion of an entire sequence, up to and including the complete sequence.

Numbering of a given amino acid or nucleotide polymer “corresponds to numbering” of a selected amino acid polymer or nucleic acid when the position of any given polymer component (amino acid residue, incorporated nucleotide, etc.) is designated by reference to the same residue position in the selected amino acid or nucleotide, rather than by the actual position of the component in the given polymer.

A vector is a composition for facilitating cell transduction by a selected nucleic acid, or expression of the nucleic acid in the cell. Vectors include, e.g., plasmids, cosmids, viruses, YACs, bacteria, poly-lysine, etc.

“Substantially an entire length of a polynucleotide or amino acid sequence” refers to at least about 70%, generally at least about 80%, or typically about 90% or more of a sequence.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. Fourth Edition (1998), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies in which a variable heavy and a variable light chain are joined together (directly or through a peptide linker) to form a continuous polypeptide.

A variety of additional terms are defined or otherwise characterized herein.

Polynucleotides

Triazine Hydrolase Sequences

The invention provides isolated or recombinant atrazine hydrolase polypeptides, and isolated or recombinant polynucleotides encoding the polypeptides. A small library of triazine hydrolases, i.e., about 1500 clones, were screened by high throughput mass spectrometry (See, e.g., High Throughput Mass Spectrometry, By Raillard et al., U.S. Ser. No. 09/499,525, filed Feb. 23, 2000 (Attorney Docket No. 02-029510US) for methods of performing high-throughput mass spectrometry) for activity against about 15 different triazine derivatives, covering a chemical structure space enabling investigation of leaving group chemistry as well as substrate bulkiness (See, e.g., FIG. 2).

The proteins of the present invention were screened for hydrolase activity against atrazine and other triazine derivatives as represented by

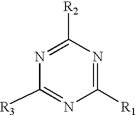

wherein the R groups are defined as described above and in FIG. 2. Examples of such triazine compounds include, but are not limited to aminotriazine, atrazine, atratone, N-methylatrazine, ametryn, aminopropazine, propazine, prometon, N-methylpropazine, prometryn, aminomorphazine, morphazine, morphatryn, morphaton, and N-methylmorphazine, which are all depicted and defined in FIG. 2. Clones were identified that showed about 2-fold to about 180 or 200-fold improvement over a wild type atrazine chlorohydrolase (as characterized at GenBank #U55933). In some embodiments, the hydrolases can display an improvement of about 500-fold or more. Clones with the ability to hydrolyze alternative substrates, i.e., other triazine derivatives, were also obtained. See, for example, Tables 3 and 4, which provide data regarding degradation of novel triazine substrates by the triazine hydrolases of the invention. Triazine hydrolases were screened for hydrolyzed product formation in the following reaction conditions: 20 μl of washed and induced cells (OD600=3) in a total volume of 100 μl 10 mM NH₃ Acetate at pH 6.8 with 250 μM substrate, i.e., triazine derivatives, at 22° C. or 37° C. Results are provided in Tables 3 and 4, provided herein.

Modified amino acid at positions 84 and 92 can affect the bulk of side chain R1 that can be accepted. For example, using site directed mutagenesis, a novel triazine hydrolase was created with an alanine at position 92 in combination with asparagine and serine at positions 328 and 331, respectively. This novel hydrolase showed enhanced dechlorination activity versus bulkier substrates.

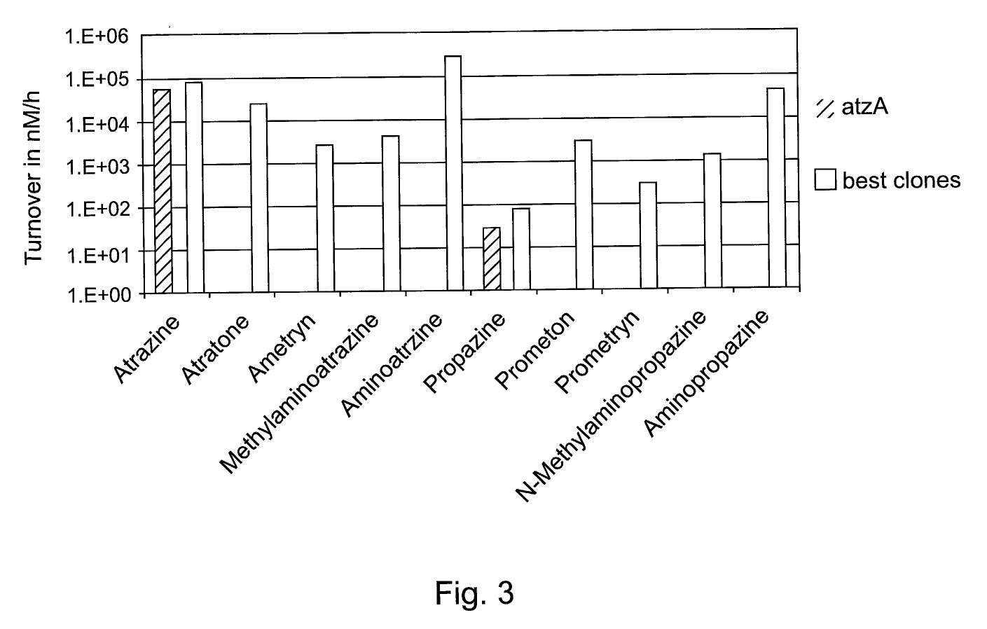

FIG. 3 provides a turnover rate in nM substrate/h/20 μl cells (at A600=3.0) for preferred triazine hydrolase library members as compared to naturally occurring atrazine chlorohydrolase (atzA) for various substrates as indicated in the figure. Novel triazine hydrolases were found that that yielded a higher transformation rate, e.g., up to 150-fold greater, than native hydrolases. In addition, the novel triazine hydrolases provided herein also hydrolyzed at least five triazine compounds, e.g., prometon, prometryn, N-methylaminopropazine, morphazine, and aminomorphazine, that were not hydrolyzed by a native hydrolase.

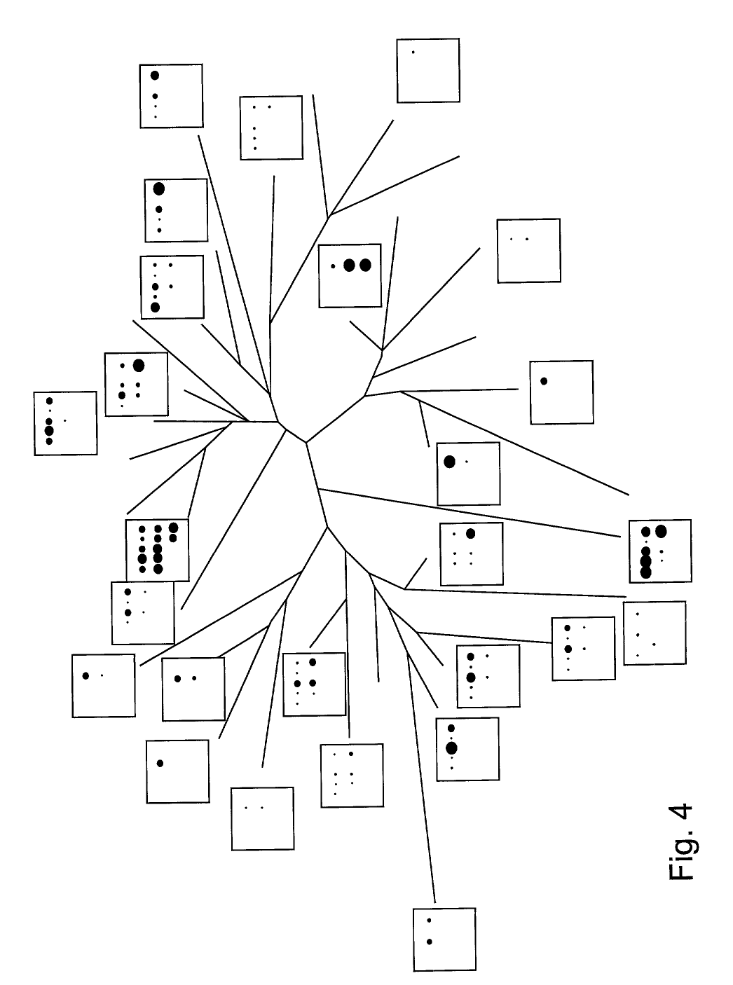

FIG. 4 provides a distribution of functional activity ion sequence space. Triazine hydrolase activity toward each of the 15 substrates provided in FIG. 2 is indicated by a circle whose area is proportional to the activity. The 15 substrates are arranged in a grid that shows relationships between enzyme sequences.

Exemplary nucleic acids that encode polypeptides having improved or expanded triazine degradation properties, such as degradation activity with respect to a variety of triazine derivatives, are provided in SEQ ID NO: 1 to SEQ ID NO: 48 encoding the polypeptides provided in SEQ ID NO: 49 to SEQ ID NO: 96. Additional triazine degrading polypeptides are exemplified by SEQ ID NOs: 97-608, as shown in Table 5. The sequences listed in Table 5, e.g., SEQ ID NOs: 97-608, are based on the polypeptide sequence of atrazine hydrolase (atzA) (Genbank #U55933), with corresponding numbering. The atzA sequence is modified as shown by the table to provide new sequences. Modifications are indicated at the positions indicated by the column headings using single letter symbols for each amino acid. For example SEQ ID NO: 97 comprises a phenylalanine at position 84, a leucine at position 92, an aspartic acid residue at position number 125, an isoleucine at position number 217, a proline residue at position 219, an isoleucine at position number 253, a glycine at position 255, an aspartic acid at position 328, and a cysteine at position 331.

Making Polynucleotides

Polynucleotides and oligonucleotides of the invention can be prepared by standard solid-phase methods, according to known synthetic methods. Typically, fragments of up to about 100 bases are individually synthesized, then joined (e.g., by enzymatic or chemical ligation methods, or polymerase mediated recombination methods) to form essentially any desired continuous sequence. For example, the polynucleotides and oligonucleotides of the invention can be prepared by chemical synthesis using, e.g., the classical phosphoramidite method described by Beaucage et al., (1981) Tetrahedron Letters 22:1859-69, or the method described by Matthes et al., (1984) EMBO J. 3: 801-05., e.g., as is typically practiced in automated synthetic methods. According to the phosphoramidite method, oligonucleotides are synthesized, e.g., in an automatic DNA synthesizer, purified, annealed, ligated and cloned in appropriate vectors.

In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (pkim@ccnet.com), HTI Bio-products, inc. (http:/www.htibio.com), BMA Biomedicals Ltd (U.K.), Bio.Synthesis, Inc., and many others.

Certain polynucleotides of the invention may also obtained by screening cDNA libraries using oligonucleotide probes which can hybridize to or PCR-amplify polynucleotides which encode the triazine hydrolase polypeptides and fragments of those polypeptides. Procedures for screening and isolating cDNA clones are well-known to those of skill in the art. Such techniques are described in, for example, Sambrook et al. (1989) supra, and Ausubel F M et al. (1989; supplemented through 1999) supra.

As described in more detail herein, the polynucleotides of the invention include sequences which encode novel triazine hydrolases and sequences complementary to the coding sequences, and novel fragments of coding sequence and complements thereof. The polynucleotides can be in the form of RNA or in the form of DNA, and include mRNA, cRNA, synthetic RNA and DNA, and cDNA. The polynucleotides can be double-stranded or single-stranded, and if single-stranded, can be the coding strand or the non-coding (anti-sense, complementary) strand. The polynucleotides optionally include the coding sequence of a triazine hydrolase (i) in isolation, (ii) in combination with additional coding sequence(s), so as to encode, e.g., a fusion protein, a pre-protein, a prepro-protein, or the like, (iii) in combination with non-coding sequences, such as introns, control elements such as a promoter, a terminator element, or 5′ and/or 3′ untranslated regions effective for expression of the coding sequence in a suitable host, and/or (iv) in a vector or host environment in which the triazine hydrolase coding sequence is a heterologous gene. Sequences can also be found in combination with typical compositional formulations of nucleic acids, including in the presence of carriers, buffers, adjuvants, excipients, vectors, vector components, and the like.

Using Polynucleotides

The polynucleotides of the invention have a variety of uses in, for example: recombinant production (i.e., expression) of the triazine hydrolase polypeptides of the invention; as soil or water treatment compositions, e.g., to encode enzymes which degrade triazine, atrazine or other triazine derivatives; as diagnostic probes for the presence of complementary or partially complementary nucleic acids (including for detection of natural triazine hydrolase coding nucleic acids); as substrates for further reactions, e.g., shuffling reactions or mutation reactions to produce new and/or improved triazine hydrolase homologues, and the like.

Expression of Polypeptides

In accordance with the present invention, polynucleotide sequences which encode novel triazine hydrolases, fragments of triazine hydrolase fusion proteins, or functional equivalents thereof, collectively referred to herein as “triazine hydrolase polypeptides,” or “triazine hydrolases,” are used in recombinant DNA molecules that direct the expression of the triazine hydrolase polypeptides in appropriate host cells. Due to the inherent degeneracy of the genetic code, other nucleic acid sequences which encode substantially the same or a functionally equivalent amino acid sequence are also used to clone and express the triazine hydrolases.

Modified Coding Sequences:

As will be understood by those of skill in the art, it can be advantageous to modify a coding sequence to enhance its expression in a particular host. The genetic code is redundant, with 64 possible codons, but most organisms preferentially use a subset of these codons. The codons that are utilized most often in a species are called optimal codons, and those not utilized very often are classified as rare or low-usage codons (see, e.g., Zhang S P et al. (1991) Gene 105:61-72). Codons can be substituted to reflect the preferred codon usage of the host, a process called “codon optimization” or “controlling for species codon bias.”

Optimized coding sequence containing codons preferred by a particular prokaryotic or eukaryotic host (see also, Murray, E. et al. (1989) Nuc. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (Dalphin M E et al. (1996) Nuc. Acids Res. 24: 216-218).

The polynucleotide sequences of the present invention are optionally engineered in order to alter a triazine hydrolase coding sequence, for a variety of reasons, including, but not limited to, alterations which modify the cloning, processing and/or expression of the gene product. For example, alterations may be introduced using techniques which are well known in the art, e.g., site-directed mutagenesis or de novo synthesis, to insert new restriction sites, to alter glycosylation patterns, to change codon preference, to introduce splice sites, etc.

Vectors, Promoters and Expression Systems,

The present invention also includes recombinant constructs comprising one or more of the nucleic acid sequences as broadly described above. The constructs comprise a vector, such as, a plasmid, a cosmid, a phage, a virus, a bacterial artificial chromosome (BAC), a yeast artificial chromosome (YAC), an agrobacterium, or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts which describe molecular biological techniques useful herein, including the use of vectors, promoters, prokaryotic and eukaryotic cell cloning, and many other relevant topics, include Berger and Kimmel, Guide to Molecular Cloning Techniques Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the homologous nucleic acids of the invention are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

The present invention also relates to host cells which are transduced with vectors of the invention, and the production of polypeptides of the invention by recombinant techniques. Host cells are genetically engineered (i.e., transduced, transformed or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants, or amplifying the triazine hydrolase gene. The culture conditions, such as temperature, pH and the like, are those previously used with the host cell selected for expression, and will be apparent to those skilled in the art and in the references cited above. Additional useful references for cloning and culture of cells include, including, e.g., Freshney (1994) Culture of Animal Cells, a Manual of Basic Technique, third edition, Wiley-Liss, New York and the references cited therein, Payne et al. (1992) Plant Cell and Tissue Culture in Liquid Systems John Wiley & Sons, Inc. New York, N.Y., and Atlas and Parks (eds) The Handbook of Microbiological Media (1993) CRC Press, Boca Raton, Fla.

The triazine hydrolase proteins of the invention can also be produced in non-animal cells such as plants, yeast, fungi, bacteria and the like. For example, bacteria expressing the polypeptides of the invention are optionally used to degrade triazine compounds, e.g., in triazine contaminated water or soil. In addition to Sambrook, Berger and Ausubel, details regarding plant cell culture can be found in Gamborg and Phillips (eds) (1995) Plant Cell, Tissue and Organ Culture; Fundamental Methods Springer Lab Manual, Springer-Verlag (Berlin Heidelberg New York).

The polynucleotides of the present invention may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses, agrobacterium, and many others. Any vector that transduces genetic material into a cell, and, if replication is desired, which is replicable and viable in the relevant host can be used depending on where expression is desired.

The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda P_{L} promoter, and other promoters known to control expression of genes in prokaryotic or eukaryotic cells or their viruses. The expression vector also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

The vector containing the appropriate DNA sequence as described above, as well as an appropriate promoter or control sequence, may be employed to transform an appropriate host to permit the host to express the protein. Examples of appropriate expression hosts especially include bacterial cells, such as E. coli, Streptomyces, and Salmonella typhimurium; as well as fungal cells, such as Saccharomyces cerevisiae, Pichiapastoris, and Neurospora crassa; insect cells such as Drosophila and Spodoptera frugiperda; mammalian cells; plant cells, etc. It is understood that not all cells or cell lines need to be capable of producing fully functional triazine hydrolase; for example, antigenic fragments of a triazine hydrolase are optionally produced in a bacterial or other expression system for generation of useful antibodies, as described in more detail below. The invention is not limited by the host cells employed.

In bacterial systems, a number of expression vectors may be selected depending upon the use intended for the triazine hydrolase. For example, when large quantities of triazine hydrolase or fragments thereof are needed for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the triazine hydrolase coding sequence is optionally ligated into the vector in-frame with sequences for the amino-terminal Met and the subsequent 7 residues of beta-galactosidase so that a hybrid protein is produced; pIN vectors (Van Heeke & Schuster (1989) J Biol Chem 264:5503-5509); pET vectors (Novagen, Madison Wis.); and the like.

In certain embodiments of the present invention, chimeric nucleic acids or other sequences are introduced into the cells of particular organisms of interest. There are several well-known methods of introducing target nucleic acids into, e.g., bacterial cells, any of which may be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention.

Bacteria are typically grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress Expression System™ from Qiagen). The isolated and purified plasmids are then further manipulated to produce other plasmids.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC.

Additional basic procedures for sequencing, cloning and other aspects of molecular biology and underlying theoretical considerations are also found in Watson et al. (1992) Recombinant DNA Second Edition Scientific American Books, NY. Furthermore, a wide variety of cloning kits and associated products are commercially available from, e.g., Pharmacia Biotech, Stratagene, Sigma-Aldrich Co., Novagen, Inc., Fermentas, and 5 Prime→3 Prime, Inc.

Similarly, in the yeast Saccharomyces cerevisiae a number of vectors containing constitutive or inducible promoters such as alpha factor, alcohol oxidase and PGH may be used for production of the triazine hydrolase proteins of the invention. For reviews, see Ausubel et al. (supra) and Grant et al. (1987; Methods in Enzymology 153:516-544).

Additional Expression Elements

Specific initiation signals can aid in efficient translation of a triazine hydrolase coding sequence. These signals can include, e.g., the ATG initiation codon and adjacent sequences. In cases where a triazine hydrolase coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon can be provided. The initiation codon is provided in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (See also, Scharf D et al. (1994) Results Probl Cell Differ 20:125-62 and Bittner et al. (1987) Methods in Enzymol 153:516-544).

Secretion/Localization Sequences

Polynucleotides of the invention can also be fused, for example, in-frame to a nucleic acid encoding a secretion/localization sequence, to target polypeptide expression to a desired cellular compartment, membrane, or organelle, or to direct polypeptide secretion to the periplasmic space or into the cell culture media. Such sequences are known to those of skill, and include secretion leader peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

Expression Hosts

In a further embodiment, the present invention relates to host cells containing the above-described constructs. The host cell can be a eukaryotic cell, such as a mammalian cell, a yeast cell, or a plant cell, or the host cell can be a prokaryotic cell, such as a bacterial cell. For bioremediation, bacterial cells are often preferred. Introduction of the construct into the host cell can be effected by calcium phosphate transfection, DEAE-Dextran mediated transfection, electroporation, or other common techniques (See, e.g., Sambrook, Ausubel, Berger (all supra). See also, Davis, L., Dibner, M., and Battey, I. (1986) Basic Methods in Molecular Biology).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre” or a “prepro” form of the protein may also be important for correct insertion, folding and/or function. Different host cells such as CHO, HeLa, BHK, MDCK, 293, WI38, etc. have specific cellular machinery and characteristic mechanisms for such post-translational activities and may be chosen to ensure the correct modification and processing of the introduced, foreign protein.

For long-term, high-yield production of recombinant proteins, stable expression can be used. For example, cell lines which stably express a polypeptide of the invention are transduced using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. For example, resistant clumps of stably transformed cells can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding triazine hydrolases of the invention can be designed with signal sequences which direct secretion of the mature polypeptides through a prokaryotic or eukaryotic cell membrane, e.g., for use in bioremediation.

Additional Polypeptide Sequences

The polynucleotides of the present invention may also comprise a coding sequence fused in-frame to a marker sequence which, e.g., facilitates purification of the encoded polypeptide. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I., et al. (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the triazine hydrolase sequence is useful to facilitate purification.

One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281) while the enterokinase cleavage site provides a means for separating the triazine hydrolase polypeptide from the fusion protein. pGEX vectors (Promega; Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Polypeptide Production and Recovery

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

As noted, many references are available for the culture and production of many cells, including cells of bacterial, plant, animal (especially mammalian) and archebacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In vitro Cell Dev. Biol. 25:1016-1024. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Plant Molecular Biolgy (1993) R. R. D. Croy, Ed. Bios Scientific Publishers, Oxford, U.K. ISBN 0 12 198370 6. Cell culture media in general are set forth in Atlas and Parks (eds) The Handbook of Microbiological Media (1993) CRC Press, Boca Raton, Fla. Additional information for cell cultures is found in available commercial literature such as the Life Science Research Cell Culture Catalogue (1998) from Sigma-Aldrich, Inc (St Louis, Mo.) (“Sigma-LSRCCC”) and, e.g., the Plant Culture Catalogue and supplement (1997) also from Sigma-Aldrich, Inc (St Louis, Mo.) (“Sigma-PCCS”).

Polypeptides of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted supra, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

In vitro Expression Systems

Cell-free transcription/translation systems can also be employed to produce polypeptides using DNAs or RNAs of the present invention. Several such systems are commercially available. A general guide to in vitro transcription and translation protocols is found in Tymms (1995) In vitro Transcription and Translation Protocols: Methods in Molecular Biology Volume 37, Garland Publishing, NY.

Modified Amino Acids

Polypeptides of the invention may contain one or more modified amino acid. The presence of modified amino acids may be advantageous in, for example, (a) increasing polypeptide serum half-life, (b) reducing polypeptide antigenicity, and (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means.

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like. References adequate to guide one of skill in the modification of amino acids are replete throughout the literature. Example protocols are found in Walker (1998) Protein Protocols on CD-ROM Human Press, Towata, N.J.

Use as Probes

Also contemplated are uses of polynucleotides, also referred to herein as oligonucleotides, typically having at least 12 bases, preferably at least 15, more preferably at least about 20, about 30, about 50 bases, or about 75 bases or more, which hybridize under highly stringent conditions to a triazine hydrolase polynucleotide as described above. The polynucleotides are optionally used as probes, primers, sense and antisense agents, and the like, according to methods as noted supra.

Sequence Variations

Silent Variations

It will be appreciated by those skilled in the art that due to the degeneracy of the genetic code, a multitude of nucleic acid sequences encoding triazine hydrolase polypeptides of the invention are optionally produced, some of which may bear minimal sequence homology to the nucleic acid sequences explicitly disclosed herein.

TABLE 1
Codon Table

| Amino acids | | | Codon |
|---|---|---|---|
| Alanine | Ala | A | GCA GCC GCG GCU |
| Gysteine | Cys | C | UGC UGU |
| Aspartic acid | Asp | D | GAC GAU |
| Glutaniic acid | Glu | E | GAA GAG |
| Phenylalanine | Phe | F | UUC UUU |
| Glycine | Gly | G | GGA GGG GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAG UAU |

For instance, inspection of the codon table (Table 1) shows that codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

Using, as an example, the nucleic acid sequence corresponding to nucleotides 1-15 of SEQ ID NO: 1, ATG CAA ACG CTC AGC, a silent variation of this sequence includes ATG CAG ACC TTA AGT, both sequences which encode the amino acid sequence MQTLS, corresponding to amino acids 1-5 of SEQ ID NO:49.

Such “silent variations” are one species of “conservatively modified variations”, discussed below. One of skill will recognize that each codon in a nucleic acid (except AUG, which is ordinarily the only codon for methionine) can be modified by standard techniques to encode a functionally identical polypeptide. Accordingly, each silent variation of a nucleic acid which encodes a polypeptide is implicit in any described sequence. The invention provides each and every possible variation of nucleic acid sequence encoding a polypeptide of the invention that could be made by selecting combinations based on possible codon choices. These combinations are made in accordance with the standard triplet genetic code (e.g., as set forth in Table 1) as applied to the nucleic acid sequence encoding a triazine hydrolase polypeptide of the invention. All such variations of every nucleic acid herein are specifically provided and described by consideration of the sequence in combination with the genetic code.

Conservative Variations

“Conservatively modified variations” or, simply, “conservative variations” of a particular nucleic acid sequence refers to those nucleic acids which encode identical or essentially identical amino acid sequences, or, where the nucleic acid does not encode an amino acid sequence, to essentially identical sequences. One of skill will recognize that individual substitutions, deletions, or additions which alter, add, or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

Conservative substitution tables providing functionally similar amino acids are well known in the art. Table 2 sets forth six groups which contain amino acids that are “conservative substitutions” for one another.

TABLE 2
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Thus, “conservatively substituted variations” of a listed polypeptide sequence of the present invention include substitutions of a small percentage, typically less than 5%, more typically less than 2% or 1%, of the amino acids of the polypeptide sequence, with a conservatively selected amino acid of the same conservative substitution group.

For example, a conservatively substituted variation of the polypeptide identified herein as SEQ ID NO:49 will contain “conservative substitutions”, according to the six groups defined above, in up to 23 residues (i.e., 5% of the amino acids) in the 474 amino acid polypeptide.

In a further example, if four conservative substitutions were localized in the region corresponding to amino acids 70-81 of SEQ ID NO:49, examples of conservatively substituted variations of this region,

NQI LLR GGP SHG include:

NNI LLK GGP AHG and

QQL IMR GGP THG and the like, in accordance with the conservative substitutions listed in Table 2 (in the above example, conservative substitutions are underlined). Listing of a protein sequence herein, in conjunction with the above substitution table, provides an express listing of all conservatively substituted proteins.

Finally, the addition of sequences which do not alter the encoded activity of a nucleic acid molecule, such as the addition of a non-functional sequence, is a conservative variation of the basic nucleic acid.

One of skill will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

Nucleic Acid Hybridization

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, N.Y.), as well as in Ausubel, supra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

“Stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen (1993), supra. and in Hames and Higgins, 1 and 2.

For purposes of the present invention, generally, “highly stringent” hybridization and wash conditions are selected to be about 5° C. lower than the thermal melting point (Tₘ) for the specific sequence at a defined ionic strength and pH. The Tₘ is the temperature (under defined ionic strength and pH) at which 50% of the test sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or Northern blot is 50% formalin with 1 mg of heparin at 42° C., with the hybridization being carried out overnight. An example of stringent wash conditions is a 0.2× SSC wash at 65° C. for 15 minutes (see, Sambrook, supra for a description of SSC buffer). Often the high stringency wash is preceded by a low stringency wash to remove background probe signal. An example low stringency wash is 2× SSC at 40° C. for 15 minutes. In general, a signal to noise ratio of 5× (or higher) than that observed for an unrelated probe in the particular hybridization assay indicates detection of a specific hybridization.

Comparative hybridization can be used to identify nucleic acids of the invention, and this comparative hybridization method is a preferred method of distinguishing nucleic acids of the invention.

In particular, detection of highly stringent hybridization in the context of the present invention indicates strong structural similarity to, e.g., the nucleic acids provided in the sequence listing herein. For example, it is desirable to identify test nucleic acids which hybridize to the exemplar nucleic acids herein under stringent conditions. One measure of stringent hybridization is the ability to hybridize to one of the listed nucleic acids, e.g., nucleic acid sequences SEQ ID NO: 1 to SEQ ID NO:48 and complementary polynucleotide sequences thereof, under highly stringent conditions. Stringent hybridization and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, the hybridization and wash conditions are gradually increased until a probe comprising one or more nucleic acid sequences selected from SEQ ID NO: 1 to SEQ ID NO:48 and complementary polynucleotide sequences thereof, binds to a perfectly matched complementary target (again, a nucleic acid comprising one or more nucleic acid sequences selected from SEQ ID NO:1 to SEQ ID NO:48 and complementary polynucleotide sequences thereof), with a signal to noise ratio that is at least 5× as high as that observed for hybridization of the probe to an unmatched target. In this case, the unmatched target is a nucleic acid corresponding to a known triazine hydrolase, e.g., a triazine hydrolase nucleic acid (other than those in the accompanying sequence listing) that is present in a public database such as GenBank™ at the time of filing of the subject application. An example of such an unmatched target nucleic acid includes, e.g., the nucleic acids corresponding to GenBank accession number: U55933 and AF312304. Additional such sequences can be identified in GenBank by one of skill.

A test nucleic acid is said to specifically hybridize to a probe nucleic acid when it hybridizes at least half as well to the probe as to the perfectly matched complementary target, i.e., with a signal to noise ratio at least half as high as hybridization of the probe to the target under conditions in which the perfectly matched probe binds to the perfectly matched complementary target with a signal to noise ratio that is at least about 3×-10× as high as that observed for hybridization to any of the unmatched target nucleic acids, such as U55933 or AF312304.

Ultra high-stringency hybridization and wash conditions are those in which the stringency of hybridization and wash conditions are increased until the signal to noise ratio for binding of the probe to the perfectly matched complementary target nucleic acid is at least 5× as high as that observed for hybridization to any of the unmatched target nucleic acids, such as U55933 or AF312304. A target nucleic acid which hybridizes to a probe under such conditions, with a signal to noise ratio of at least half that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or even 500× or more as high as that observed for hybridization to any of the unmatched target nucleic acids (U55933) can be identified. A target nucleic acid which hybridizes to a probe under such conditions, with a signal to noise ratio of at least half that of the perfectly matched complementary target nucleic acid is said to bind to the probe under ultra-ultra-high stringency conditions.

Target nucleic acids which hybridize to the nucleic acids represented by SEQ ID NO:1 to SEQ ID NO:48 under high, ultra-high and ultra-ultra high stringency conditions are a feature of the invention. Examples of such nucleic acids include those with one or a few silent or conservative nucleic acid substitutions as compared to a given nucleic acid sequence.

Nucleic acids which do not hybridize to each other under stringent conditions are still substantially identical if the polypeptides which they encode are substantially identical. This occurs, e.g., when a nucleic acid is created using the maximum codon degeneracy permitted by the genetic code, or when the polypeptide encoded by the nucleic acid binds to antisera generated against one or more of SEQ ID NO:49 to SEQ ID NO:608 which has been subtracted using the polypeptides encoded by an atrazine hydrolase sequence in GenBank, such as U55933. Further details on immunological identification of polypeptides of the invention are found below.

In one aspect, the invention provides a nucleic acid which comprises a unique subsequence in a nucleic acid selected from SEQ ID NO:1 to SEQ ID NO:48. The unique subsequence is unique as compared to a nucleic acid corresponding to U55933 or any other triazine hydrolase homologue nucleic acid (other than those in the accompanying sequence listing) that is present in a public database such as GenBank™ at the time of filing of the subject application. Such unique subsequences can be determined by aligning any of SEQ ID NO:1 to SEQ ID NO:48 against the complete set of nucleic acids corresponding to known atrazine or triazine hydrolases. Alignment can be performed using the BLAST algorithm set to default parameters. Any unique subsequence is useful, e.g., as a probe to identify the nucleic acids of the invention.

Similarly, the invention includes a polypeptide which comprises a unique subsequence in a polypeptide selected from: SEQ ID NO: 49 to SEQ ID NO: 608. Here, the unique subsequence is unique as compared to a polypeptide corresponding U55933, AF312304, or any other triazine hydrolase homologue nucleic acid (other than those in the accompanying sequence listing) that is present in a public database such as GenBank™ at the time of filing of the subject application (the control polypeptides) (note that where the sequence corresponds to a non-translated sequence such as a pseudo gene, the corresponding polypeptide is generated simply by in silico translation of the nucleic acid sequence into an amino acid sequence, where the reading frame is selected to correspond to the reading frame of homologous triazine hydrolase nucleic acids.

The invention also provides for target nucleic acids that hybridize under stringent conditions to a unique coding oligonucleotide which encodes a unique subsequence in a polypeptide selected from: SEQ ID NO:49 to SEQ ID NO:608, wherein the subsequence is unique as compared to a polypeptide corresponding to any of the control polypeptides, e.g., the polypeptide encoded by the nucleic acid represented by U55933, AF312304, or any other triazine hydrolase nucleic acid that is present in a public database such as GenBank™ at the time of filing of the subject application. Unique sequences are determined as noted above.

In one example, the stringent conditions are selected such that a perfectly complementary oligonucleotide to the coding oligonucleotide hybridizes to the coding oligonucleotide with at least about a 5-10× higher signal to noise ratio than for hybridization of the perfectly complementary oligonucleotide to a control nucleic acid corresponding to any of the control polypeptides. Conditions can be selected such that higher ratios of signal to noise are observed in the particular assay which is used, e.g., about 15×, 20×, 30×, 50× or more. In this example, the target nucleic acid hybridizes to the unique coding oligonucleotide with at least a 2× higher signal to noise ratio as compared to hybridization of the control nucleic acid to the coding oligonucleotide. Again, higher signal to noise ratios can be selected, e.g., about 5×, 10×, 20×, 30×, 50× or more. The particular signal will depend on the label used in the relevant assay, e.g., a fluorescent label, a colorimetric label, a radio active label, or the like.

Substrates and Formats for Sequence Recombination

The polynucleotides of the invention are optionally used as substrates for a variety of recombination and recursive recombination (e.g., DNA shuffling) reactions and/or other diversity generating reactions, in addition to or concurrent with standard cloning methods, to produce triazine hydrolase homologues with desired properties. A variety of such reactions are known, including those developed by the inventors and their co-workers.

The following publications describe a variety of recursive recombination procedures and/or methods which can be incorporated into such procedures: Stemmer, et al., (1999) “Molecular breeding of viruses for targeting and other clinical properties. Tumor Targeting” 4:1-4; Nesset al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameriet al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proceedings of the National Academy of Sciences, U.S.A. 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proceedings of the National Academy of Sciences. U.S.A. 91:10747-10751.

Additional details regarding DNA shuffling methods are found in U.S. patents by the inventors and their co-workers, including: U.S. Pat. No. 5,605,793 to Stemmer (Feb. 25, 1997), “METHODS FOR IN VITRO RECOMBINATION;” U.S. Pat. No. 5,811,238 to Stemmer et al. (Sep. 22, 1998) “METHODS FOR GENERATING POLYNUCLEOTIDES HAVING DESIRED CHARACTERISTICS BY ITERATIVE SELECTION AND RECOMBINATION;” U.S. Pat. No. 5,830,721 to Stemmer et al. (Nov. 3, 1998), “DNA MUTAGENESIS BY RANDOM FRAGMENTATION AND REASSEMBLY;” U.S. Pat. No. 5,834,252 to Stemmer, et al. (Nov. 10, 1998) “END-COMPLEMENTARY POLYMERASE REACTION,” and U.S. Pat. No. 5,837,458 to Minshull, et al. (Nov. 17, 1998), “METHODS AND COMPOSITIONS FOR CELLULAR AND METABOLIC ENGINEERING.”

In addition, details and formats for DNA shuffling are found in a variety of PCT and foreign patent application publications, including: Stemmer and Crameri, “DNA MUTAGENESIS BY RANDOM FRAGMENTATION AND REASEMBLY” WO 95/22625; Stemmer and Lipschutz “END COMPLEMENTARY POLYMERASE CHAIN REACTION” WO 96/33207; Stemmer and Crameri “METHODS FOR GENERATING POLYNUCLEOTIDES HAVING DESIRED CHARACTERISTICS BY ITERATIVE SELECTION AND RECOMBINATION” WO 97/0078; Minshull and Stemmer, “METHODS AND COMPOSITIONS FOR CELLULAR AND METABOLIC ENGINEERING” WO 97/35966; Punnonen et al. “TARGETING OF GENETIC VACCINE VECTORS” WO 99/41402; Punnonen et al. “ANTIGEN LIBRARY IMMUNIZATION” WO 99/41383; Punnonen et al. “GENETIC VACCINE VECTOR ENGINEERING” WO 99/41369; Punnonen et al. OPTIMIZATION OF IMMUNODULATORY PROPERTIES OF GENETIC VACCINES WO 9941368; Stemmer and Crameri, “DNA MUTAGENESIS BY RANDOM FRAGMENTATION AND REASSEMBLY” EP 0934999; Stemmer “EVOLVING CELLULAR DNA UPTAKE BY RECURSIVE SEQUENCE RECOMBINATION” EP 0932670; Stemmer et al., “MODIFICATION OF VIRUS TROPISM AND HOST RANGE BY VIRAL GENOME SHUFFLING” WO 9923107; Apt et al., “HUMAN PAPILLOMAVIRUS VECTORS” WO 9921979; Del Cardayre et al. “EVOLUTION OF WHOLE CELLS AND ORGANISMS BY RECURSIVE SEQUENCE RECOMBINATION” WO 9831837; Patten and Stemmer, “METHODS AND COMPOSITIONS FOR POLYPEPTIDE ENGINEERING” WO 9827230; Stemmer et al., and “METHODS FOR OPTIMIZATION OF GENE THERAPY BY RECURSIVE SEQUENCE SHUFFLING AND SELECTION” WO9813487.

Certain U.S. Applications provide additional details regarding DNA shuffling and related techniques, including “SHUFFLING OF CODON ALTERED GENES” by Patten et al. filed September 29, 1998, (U.S. Ser. No. 60/102,362), Jan. 29, 1999 (U.S. Ser. No. 60/117,729), and Sep. 28, 1999, U.S. Ser. No. 09/22588 (Attorney Docket Number 20-28520US/PCT); “EVOLUTION OF WHOLE CELLS AND ORGANISMS BY RECURSIVE SEQUENCE RECOMBINATION”, by del Cardyre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Feb. 5, 1999 (U.S. Ser. No. 60/118,813) and filed Jun. 24, 1999 (U.S. Ser. No. 60/141,049) and filed Sep. 28, 1999 (U.S. Ser. No. 09/408,392, Attorney Docket Number 02-29620US); and “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393, Attorney Docket Number 02-010070US); and “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118854, U.S. Ser. No. 09/416,375 and U.S. Ser. No. 09/494,282).

As review of the foregoing publications, patents, published applications and U.S. patent applications reveals, shuffling (or “recursive recombination”) of nucleic acids to provide new nucleic acids with desired properties can be carried out by a number of established methods. Any of these methods can be adapted to the present invention to evolve the triazine hydrolases discussed herein to produce new triazine hydrolases with improved properties. Both the methods of making such hydrolases and the hydrolases produced by these methods are a feature of the invention.

In brief, at least five different general classes of recombination methods are applicable to the present invention. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined in vivo, e.g., by allowing recombination to occur between nucleic acids in cells. Third, whole cell genome recombination methods can be used in which whole genomes of cells are recombined, optionally including spiking of the genomic recombination mixtures with desired library components such as triazine hydrolase nucleic acids. Fourth, synthetic recombination methods can be used, in which oligonucleotides corresponding to different triazine hydrolases are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made by tri-nucleotide synthetic approaches. Fifth, in silico methods of recombination can be effected in which genetic algorithms are used in a computer to recombine sequence strings which correspond to triazine hydrolases such as those listed in the sequence listing herein. The resulting recombined sequence strings are optionally converted into nucleic acids by synthesis of nucleic acids which correspond to the recombined sequences, e.g., in concert with oligonucleotide synthesis/gene reassembly techniques. Any of the preceding general recombination formats are optionally practiced in a reiterative fashion to generate a more diverse set of recombinant nucleic acids.

The above references provide these and other basic recombination formats as well as many modifications of these formats. Regardless of the format that is used, the nucleic acids of the invention are optionally recombined (with each other or with related (or even unrelated) nucleic acids) to produce a diverse set of recombinant nucleic acids, including homologous nucleic acids. In general, the sequence recombination techniques described herein provide particular advantages in that they provide for recombination between the nucleic acids of SEQ ID NO:1 to SEQ ID NO:48 or derivatives thereof, in any available format, thereby providing a very fast way of exploring the manner in which different combinations of sequences can affect a desired result. For example, desired results for improved triazine hydrolases include, but are not limited to, the ability to hydrolyze a different substrate, e.g., with a different leaving group or different steric hindrance properties.

Following recombination, any nucleic acids which are produced can be selected for a desired activity. In the context of the present invention, this can include testing for and identifying triazine hydrolase activities, by any of the assays in the art. In addition, useful properties such as the ability to hydrolyze a variety of substrates with a variety of leaving groups can also be selected for. A variety of triazine hydrolase related (or even unrelated) properties are optionally assayed for, using any available assay.

A recombinant nucleic acid produced by recursively recombining one or more polynucleotides of the invention with one or more additional nucleic acid also forms a part of the invention. The one or more additional nucleic acid may include another polynucleotide of the invention; optionally, alternatively, or in addition, the one or more additional nucleic acid can include, e.g., a nucleic acid encoding a naturally-occurring triazine hydrolase or a subsequence thereof, any homologous triazine hydrolase sequence or subsequence thereof, or any triazine hydrolase sequence as found in GenBank or other available literature, or, e.g., any other homologous or non-homologous nucleic acid (certain recombination formats noted above, notably those performed synthetically or in silico, do not require homology for recombination).

The recombining steps may be performed in vivo, in vitro, or in silico as described in more detail in the references above. Also included in the invention is a cell containing any resulting recombinant nucleic acid, nucleic acid libraries produced by recursive recombination of the nucleic acids set forth herein, and populations of cells, vectors, viruses, plasmids or the like comprising the library or comprising any recombinant nucleic acid resulting from recombination (or recursive recombination) of a nucleic acid as set forth herein with another such nucleic acid, or an additional nucleic acid. Corresponding sequence strings in a database present in a computer system or computer readable medium are a feature of the invention.

Other Polynucleotide Compositions

The invention also includes compositions comprising two or more polynucleotides of the invention (e.g., as substrates for recombination). The composition can comprise a library of recombinant nucleic acids, where the library contains at least 2, 3, 5, 10, 20, or 50 or more nucleic acid species. The nucleic acids are optionally cloned into expression vectors, providing expression libraries.

The invention also includes compositions produced by digesting one or more polynucleotide of the invention with a restriction endonuclease, an RNAse, or a DNAse (e.g., as is performed in certain of the recombination formats noted above); and compositions produced by fragmenting or shearing one or more polynucleotide of the invention by mechanical means (e.g., sonication, vortexing, flow based fragmentation, and the like), which can also be used to provide substrates for recombination in the methods above. Similarly, compositions comprising sets of oligonucleotides corresponding to more than one nucleic acid of the invention are useful as recombination substrates and are a feature of the invention. For convenience, these fragmented, sheared, or oligonucleotide synthesized mixtures are referred to as fragmented nucleic acid sets.

Also included in the invention are compositions produced by incubating one or more of the fragmented nucleic acid sets in the presence of ribonucleotide- or deoxyribonucleotide triphosphates and a nucleic acid polymerase. This resulting composition forms a recombination mixture for many of the recombination formats noted above. The nucleic acid polymerase may be an RNA polymerase, a DNA polymerase, or an RNA-directed DNA polymerase (e.g., a “reverse transcriptase”); the polymerase can be, e.g., a thermostable DNA polymerase (such as, VENT, TAQ, or the like).

Triazine Hydrolase Polypeptides

The invention provides isolated or recombinant triazine hydrolase polypeptides, referred to herein as “triazine hydrolase polypeptides” or simply “triazine hydrolases.” An isolated or recombinant triazine hydrolase polypeptide of the invention includes a polypeptide comprising a sequence selected from SEQ ID NO:49 to SEQ ID NO:608 and conservatively modified variants thereof.

Several conclusions may be drawn from comparison of the exemplary sequences of the invention to sequences of known, naturally-occurring triazine hydrolases, such as atrazine chlorohydrolase U55933. Such sequences are readily available from a variety of sources, such as GenBank, and the Pfam (Protein Families) database at http://www.sanger.ac.uk/Software/Pfam/index.shtml

Of particular note is the presence of differing amino acids in some triazine hydrolase polypeptide sequences of the invention at the following positions: 84, 92, 125, 217, 219, 253, 255, 328, and 331 (corresponding to nucleic acid positions 250, 274, 375, 650, 655, 757, 763, 982, and 991). In other words, amino acid residues in these positions are typically different in the improved hydrolases of the invention as compared to the equivalent position of known, naturally-occurring or recombinant triazine hydrolase sequences, i.e., U55933. The triazine hydrolases of the present invention with variations at these positions exhibit improved activity as compared with atrazine chlorohydrolase U55933 or activity against a novel or alternative substrate. Other unique (as compared to U55933) amino acid residues present in some of the polypeptides of the invention correspond to amino acid positions 30, 160, 386, and 465 (nucleic acid positions 89, 478, 1157, and 1395). All numbering is in relation to the nucleic acid of SEQ ID NO: 1 or the amino acid sequence of SEQ ID NO: 49.

The invention includes a triazine hydrolase polypeptide comprising at least about 20 or at least about 50 contiguous amino acids of any one of SEQ ID NO:49-608, and one or more amino acid at position 84, 92, 125, 217, 219, 253, 255, 328, and 331 that is unique as compared to U55933 or AF312304, wherein the numbering of the amino acids corresponds to that of SEQ ID NO:49.

In other embodiments, the invention includes a triazine hydrolase polypeptide that is at least about 70% homologous to SEQ ID NO: 49, wherein position number 84 comprises leucine or phenylalanine, position number 92 comprises a leucine, valine or alanine residue, position number 125 comprises glutamic acid, position number 217 comprises threonine, position number 219 comprises threonine, position number 253 comprises leucine or isoleucine, position number 255 comprises glycine or tryptophan, position number 328 comprises aspartic acid or asparagine, and position number 331 comprises serine or cysteine and wherein the polypeptide is unique as compared to atrazine chlorohydrolase (atzA, U55933) or triazine hydrolase (AF312304).

For example, preferred polypeptides of the present invention include, but are not limited to, modified versions of atzA (U55933) or AF312304, wherein the modified sequences comprise one or more modification selected from: L₈₄, L₉₂, D₁₂₅, I₂₁₇, P₂₁₉, L₂₅₃, W₂₅₅, D₃₂₈, and C₃₃₁. All numbering corresponds to SEQ ID NO: 49. These polypeptides typically comprise a triazine hydrolase activity, such as activity toward atrazine, atratone, or the like. Example modifications include, the following:

L₉₂, E₁₂₅, T₂₁₇, P₂₁₉, L₂₅₃, W₂₅₅, and S₃₃₁;
L₉₂, E₁₂₅, T₂₁₇, P₂₁₉, L₂₅₃, G₂₅₅, and S₃₃₁;
V₉₂, E₁₂₅, T₂₁₇, P₂₁₉, L₂₅₃, W₂₅₅, and S₃₃₁;
V₉₂, E₁₂₅, T₂₁₇, P₂₁₉, L₂₅₃, G₂₅₅, and S₃₃₁;
L₉₂, E₁₂₅, T₂₁₇, P₂₁₉, L₂₅₃, W₂₅₅, and S₃₃₁;
L₉₂, E₁₂₅, T₂₁₇, P₂₁₉, L₂₅₃, G₂₅₅, and S₃₃₁;
V₉₂, E₁₂₅, I₂₁₇, P₂₁₉, L₂₅₃, W₂₅₅, and S₃₃₁;
V₉₂, E₁₂₅, I₂₁₇, P₂₁₉, L₂₅₃, G₂₅₅, and S₃₃₁;
L₉₂, D₁₂₅, T₂₁₇, P₂₁₉, L₂₅₃, G₂₅₅, and S₃₃₁;
V₉₂, E₁₂₅, T₂₁₇, P₂₁₉, L₂₅₃, W₂₅₅, and C₃₃₁;
V₉₂, E₁₂₅, T₂₁₇, P₂₁₉, L₂₅₃, G₂₅₅, and C₃₃₁;
V₉₂, E₁₂₅, T₂₁₇, P₂₁₉, L₂₅₃, W₂₅₅, and S₃₃₁;
L₉₂, E₁₂₅, T₂₁₇, T₂₁₉, L₂₅₃, W₂₅₅, and S₃₃₁;
V₉₂, E₁₂₅, T₂₁₇, P₂ₗ₉, L₂₅₃, G₂₅₅, and S₃₃₁;
L₉₂, E₁₂₅, T₂₁₇, T₂₁₉, L₂₅₃, G₂₅₅, and S₃₃₁;
V₉₂, D₁₂₅, T₂₁₇, P₂₁₉, L₂₅₃, W₂₅₅, and S₃₃₁;
V₉₂, D₁₂₅, T₂₁₇, P₂₁₉, L₂₅₃, G₂₅₅, and S₃₃₁;
V₉₂, E₁₂₅, T₂₁₇, T₂₁₉, L₂₅₃, W₂₅₅, and S₃₃₁;
V₉₂, E₁₂₅, T₂₁₇, T₂₁₉, L₂₅₃, G₂₅₅, and S₃₃₁;
F₈₄, L₉₂, E₁₂₅, L₂₅₃, G₂₅₅, D₃₂₈, and C₃₃₁;
F₈₄, L₉₂, E₁₂₅, I₂₅₃, G₂₅₅, D₃₂₈, and C₃₃₁;
F₈₄, L₉₂, E₁₂₅, L₂₅₃, W₂₅₅, D₃₂₈, and C₃₃₁;
F₈₄, L₉₂, E₁₂₅, I₂₅₃, W₂₅₅, D₃₂₈, and C₃₃₁;
L₈₄, L₉₂, E₁₂₅, L₂₅₃, G₂₅₅, D₃₂₈, and C₃₃₁;
L₈₄, L₉₂, E₁₂₅, I₂₅₃, G₂₅₅, D₃₂₈, and C₃₃₁;
F₈₄, L₉₂, D₁₂₅, L₂₅₃, G₂₅₅, D₃₂₈, and C₃₃₁;
F₈₄, L₉₂, D₁₂₅, I₂₅₃, G₂₅₅, D₃₂₈, and C₃₃₁;
L₈₄, L₉₂, E₁₂₅, L₂₅₃, W₂₅₅, D₃₂₈, and C₃₃₁;
L₈₄, L₉₂, E₁₂₅, I₂₅₃, W₂₅₅, D₃₂₈, and C₃₃₁;
F₈₄, L₉₂, D₁₂₅, L₂₅₃, W₂₅₅, D₃₂₈, and C₃₃₁;
F₈₄, L₉₂, D₁₂₅, I₅₃, W2₅₅, D₃₂₈, and C₃₃₁;
L₈₄, L₉₂, D₁₂₅, L₂₅₃, G₂₅₅, D₃₂₈, and C₃₃₁;
L₈₄, L₉₂, D₁₂₅, L₂₅₃, G₂₅₅, D₃₂₈, and C₃₃₁;
L₈₄, L₉₂, D₁₂₅, L₂₅₃, W₂₅₅, D₃₂₈, and C₃₃₁; and,
L₈₄, L₉₂, D₁₂₅, L₂₅₃, W₂₅₅, D₃₂₈, and C₃₃₁.

Preferred polypeptide sequences showing novel triazine hydrolase activity include, but are not limited to, the following polypeptides represented in Table 5. In Table 5, library members 128, 124, 256, and 252 are preferred atrazine hydrolases. Preferred hydrolases for activity toward atratone include library members 121, 113, 125, 117, 377, 369, 57, 49, 381, 373, 61, 53, 313, 305, 317, and 309. Preferred ametryn hydrolases include library members 378, 377, 382, 314, 381, 313, 379, 383, 370, 121, 318, 317, 369, 125, and 374. Preferred hydrolases having activity toward amino-atrazine include, but are not limited to, library members 309, 373, 317, 381, 305, 369, 313, and 377. Preferred prometryn hydrolases, include, but are not limited to, library members 498, 502, 506, 482, 497, 510, 501, 505, and 466. Library members 252, 244, 508, 256, 500, 248, 512, and 504 comprise preferred hydrolases for propazine substrates and library members 505, 509, 507, 506, 497, 249, and 441 are preferred for activity against amino-propazine. NME-propazine activity is represented by preferred library members 477, 473, 509, 505, 469, 465, 501, 506, and 510 in Table 5 and preferred NME-atrazine hydrolases include library members 377, 381, 505, 369, 509, 373, 121, 313, 125, 345, 317, and 349. The substrate activity column in Table 5 indicates preferred embodiments and is not exclusive.

Other modifications are exemplified by those sequences corresponding to SEQ ID NOs: 97-608. Polynucleotides that are at least about 70% identical to SEQ ID NO: 1 with the corresponding changes are also included in the present invention.

Making Polypeptides

Recombinant methods for producing and isolating triazine hydrolase polypeptides of the invention are described above. In addition to recombinant production, the polypeptides may be produced by direct peptide synthesis using solid-phase techniques (cf Stewart et al. (1969) Solid-Phase Peptide Synthesis, WH Freeman Co, San Francisco; Merrifield J (1963) J. Am. Chem. Soc. 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. For example, subsequences may be chemically synthesized separately and combined using chemical methods to provide full-length triazine hydrolases.

Using Polypeptides

Antibodies

In another aspect of the invention, a triazine hydrolase polypeptide of the invention is used to produce antibodies which have, e.g., diagnostic and therapeutic uses, e.g., related to the activity, distribution, and expression of triazine hydrolases.

Antibodies to triazine hydrolases of the invention may be generated by methods well known in the art. Such antibodies may include, but are not limited to, polyclonal, monoclonal, chimeric, humanized, single chain, Fab fragments and fragments produced by an Fab expression library. Antibodies, i.e., those which block receptor binding, are especially preferred for therapeutic use.

Triazine hydrolase polypeptides for antibody induction do not require biological activity; however, the polypeptide or oligopeptide must be antigenic. Peptides used to induce specific antibodies may have an amino acid sequence consisting of at least 10 amino acids, preferably at least 15 or 20 amino acids. Short stretches of a triazine hydrolase polypeptide may be fused with another protein, such as keyhole limpet hemocyanin, and antibody produced against the chimeric molecule.

Methods of producing polyclonal and monoclonal antibodies are known to those of skill in the art, and many antibodies are available. See, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward, et al. (1989) Nature 341: 544-546. Specific monoclonal and polyclonal antibodies and antisera will usually bind with a K_{D} of at least about 0.1 μM, preferably at least about 0.01 μM or better, and most typically and preferably, 0.001 μM or better.

Detailed methods for preparation of chimeric (humanized) antibodies can be found in U.S. Pat. No. 5,482,856. Additional details on humanization and other antibody production and engineering techniques can be found in Borrebaeck (ed) (1995) Antibody Engineering, 2^{nd} Edition Freeman and Company, NY (Borrebaeck); McCafferty et al. (1996) Antibody Engineering, A Practical Approach IRL at Oxford Press, Oxford, England (McCafferty), and Paul (1995) Antibody Engineering Protocols Humana Press, Towata, N.J. (Paul).

Sequence Variations

Conservatively Modified Variations

Triazine hydrolase polypeptides of the present invention include conservatively modified variations of the sequences disclosed herein as SEQ ID NO:49 to SEQ ID NO:608. Such conservatively modified variations comprise substitutions, additions or deletions which alter, add or delete a single amino acid or a small percentage of amino acids (typically less than about 5%, more typically less than about 4%, 2%, or 1%) in any of SEQ ID NO:49 to SEQ ID NO:608.

For example, a conservatively modified variation (e.g., deletion) of the 474 amino acid polypeptide identified herein as SEQ ID NO:49 will have a length of at least about 450 amino acids, preferably at least about 455 amino acids, more preferably at least about 465 amino acids, and still more preferably at least about 470 amino acids, corresponding to a deletion of less than about 5%, 4%, 2% or 1% of the polypeptide sequence.

Another example of a conservatively modified variation (e.g., a “conservatively substituted variation”) of the polypeptide identified herein as SEQ ID NO:49 will contain “conservative substitutions”, according to the six substitution groups set forth in Table 2 (supra), in up to about 23 residues (i.e., less than about 5%) of the 474 amino acid polypeptide.

The triazine hydrolase polypeptide sequences of the invention, including conservatively substituted sequences, can be present as part of larger polypeptide sequences such as occur upon the addition of one or more domains for purification of the protein (e.g., poly his segments, FLAG tag segments, etc.), e.g., where the additional functional domains have little or no effect on the activity of the triazine hydrolase portion of the protein, or where the additional domains can be removed by post synthesis processing steps such as by treatment with a protease.

In various embodiments, the polypeptides of the invention comprise at least about 30, or at least about 50, at least about 70, at least about 100, at least about 120, at least about 150, or at least about 155 contiguous amino acid residues of any one of SEQ ID NO:49-608.

Defining Polypeptides by Immunoreactivity

Because the polypeptides of the invention provide a variety of new polypeptide sequences as compared to other triazine hydrolases, the polypeptides also provide new structural features which can be recognized, e.g., in immunological assays. The generation of antisera which specifically binds the polypeptides of the invention, as well as the polypeptides which are bound by such antisera, are a feature of the invention.

The invention includes triazine hydrolase proteins that specifically bind to or that are specifically immunoreactive with an antibody or antisera generated against an immunogen comprising an amino acid sequence selected from one or more of SEQ ID NO: SEQ ID NO: 49 to SEQ ID NO: 608. To eliminate cross-reactivity with other triazine hydrolases, the antibody or antisera is subtracted with available triazine hydrolases, such as that represented at GenBank accession numbers U55933 (a control triazine hydrolase nucleic acid). Where the accession number corresponds to a nucleic acid, a polypeptide encoded by the nucleic acid is generated and used for antibody/antisera subtraction purposes. Where the nucleic acid corresponds to a non-coding sequence, e.g., a pseudo gene, an amino acid which corresponds to the reading frame of the nucleic acid is generated (e.g., synthetically), or is minimally modified to include a start codon for recombinant production.

In one typical format, the immunoassay uses a polyclonal antiserum which was raised against one or more polypeptide comprising one or more of the sequences corresponding to one or more of: SEQ ID NO:49 to SEQ ID NO: 608 or a substantial subsequence thereof (i.e., at least about 30% of the full length sequence provided). The full set of potential polypeptide immunogens derived from SEQ ID NO:49 to SEQ ID NO: 608 are collectively referred to below as “the immunogenic polypeptides.” The resulting antisera is optionally selected to have low cross-reactivity against the control triazine hydrolases other known triazine hydrolases and any such cross-reactivity is removed by immunoabsorbtion with one or more of the control triazine hydrolases, such as atrazine chlorohydrolase, prior to use of the polyclonal antiserum in the immunoassay.

In order to produce antisera for use in an immunoassay, one or more of the immunogenic polypeptides is produced and purified as described herein. For example, recombinant protein may be produced in a mammalian cell line. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, Harlow and Lane (1988) Antibodies, A Laboratory Manual, Cold Spring Harbor Publications, New York, for a standard description of antibody generation, immunoassay formats and conditions that can be used to determine specific immunoreactivity). Alternatively, one or more synthetic or recombinant polypeptide derived from the sequences disclosed herein is conjugated to a carrier protein and used as an immunogen.

Polyclonal sera are collected and titered against the immunogenic polypeptide in an immunoassay, for example, a solid phase immunoassay with one or more of the immunogenic proteins immobilized on a solid support. Polyclonal antisera with a titer of 10⁶ or greater are selected, pooled and subtracted with the control triazine hydrolase polypeptides to produce subtracted pooled titered polyclonal antisera.

The subtracted pooled titered polyclonal antisera are tested for cross reactivity against the control triazine hydrolases. Preferably at least two of the immunogenic triazine hydrolases are used in this determination, preferably in conjunction with at least two control hydrolase homologues, to identify antibodies which are specifically bound by the immunogenic protein(s).

In this comparative assay, discriminatory binding conditions are determined for the subtracted titered polyclonal antisera which result in at least about a 5-10 fold higher signal to noise ratio for binding of the titered polyclonal antisera to the immunogenic triazine hydrolases as compared to binding to the control triazine hydrolases. That is, the stringency of the binding reaction is adjusted by the addition of non-specific competitors such as albumin or non-fat dry milk, or by adjusting salt conditions, temperature, or the like. These binding conditions are used in subsequent assays for determining whether a test polypeptide is specifically bound by the pooled subtracted polyclonal antisera. In particular, test polypeptides which show at least a 2-5× higher signal to noise ratio than the control polypeptides under discriminatory binding conditions, and at least about a one half signal to noise ratio as compared to the immunogenic polypeptide(s), shares substantial structural similarity with the immunogenic polypeptide as compared to known triazine hydrolases, and is, therefore a polypeptide of the invention.

In another example, immunoassays in the competitive binding format are used for detection of a test polypeptide. For example, as noted, cross-reacting antibodies are removed from the pooled antisera mixture by immunoabsorbtion with the control triazine hydrolase polypeptides. The immunogenic polypeptide(s) are then immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control proteins to compete for binding to the pooled subtracted antisera is determined as compared to the ability of the immunogenic polypeptide(s) to compete for binding to the antisera. Again, the percent cross-reactivity for the control polypeptides is calculated, using standard calculations. Where the percent cross-reactivity is at least 5-10× as high for the test polypeptides, the test polypeptides are said to specifically bind the pooled subtracted antisera.

In general, the immunoabsorbed and pooled antisera can be used in a competitive binding immunoassay as described herein to compare any test polypeptide to the immunogenic polypeptide(s). In order to make this comparison, the two polypeptides are each assayed at a wide range of concentrations and the amount of each polypeptide required to inhibit 50% of the binding of the subtracted antisera to the immobilized protein is determined using standard techniques. If the amount of the test polypeptide required is less than twice the amount of the immunogenic polypeptide that is required, then the test polypeptide is said to specifically bind to an antibody generated to the immunogenic protein, provided the amount is at least about 5-10× as high as for a control polypeptide.

As a final determination of specificity, the pooled antisera is optionally fully immunabsorbed with the immunogenic polypeptide(s) (rather than the control polypeptides) until little or no binding of the resulting immunogenic polypeptide subtracted pooled antisera to the immunogenic polypeptide(s) used in the immunoabsorbtion is detectable. This fully immunosorbed antisera is then tested for reactivity with the test polypeptide. If little or no reactivity is observed (i.e., no more than 2× the signal to noise ratio observed for binding of the fully immunoabsorbed antisera to the immunogenic polypeptide), then the test polypeptide is specifically bound by the antisera elicited by the immunogenic protein.

Degradation of Triazine Compounds with Triazine Hydrolases

Due to their activity against triazine derivatives, the triazine hydrolases of the invention are optionally used in compositions (in vivo or in vitro) to serve as decontamination or cleaning solutions for water or soil contaminated with atrazine or another triazine derivative, such as aminotriazine, atrazine, atratone, N-methylatrazine, ametryn, aminopropazine, propazine, prometon, N-methylpropazine, prometryn, aminomorphazine, morphazine, morphatryn, morphaton, or N-methylmorphazine, or the like (as described above).

The polypeptides presented herein provide hydrolases that degrade novel substrates in comparison to wild-type atrazine chlorohydrolase. For example, the hydrolases of the invention are optionally used to remove alternate leaving groups from triazine derivatives. For example, NH₂, Cl or other halogens, O—CH₃, —NHCH₃, —SCH₃, or the like are optionally removed from a triazine derivative by one or more of the hydrolases provided herein. In addition, substrates with greater or lesser steric hindrance are also degraded by one or more the hydrolases provided herein. Examples of substrate compounds are provided as follows:

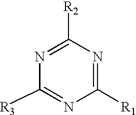

wherein R₁ and R₃ each independently comprise an amino group, i.e., —NH₂, or a substituted linear, branched, or cyclic amino group. Typically R₁ and R₃ are each independently a lower-alkyl-substituted amino group or a morpholino group. As used herein, the term “lower alkyl” refers to a C₁₋₆ alkyl. More typically, R₁ and R₃ are each independently —NH(C₂H₅), —NHCH(CH₃)₂,

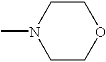

or the like. Preferably, R₃ is —NHCH(CH₃)₂, R₂ is an amino group, i.e., —NH₂, or an optionally substituted amino group, e.g., —NRH or —NRR′, a halo, a lower alkoxy, or —S—R, where R and R′ are each independently a lower alkyl group. Typically R₂ is —NH₂, —X, wherein X is a halogen such as Cl, —OCH₃, —NH(CH₃), or —S—CH₃. A larger R₁ and/or R₃ group leads to greater steric hindrance. Tables 3 and 4 provide data illustrating the use of the novel polypeptides of the invention against substrates that cannot be degraded by other triazine hydrolases including substrates such as ametryn, N-methylatrazine, prometryn, N-methylpropazine, prometon, and the like.

The present invention provides for the use of the novel triazine hydrolases of the invention in decontamination solutions, as well as such compositions containing the mutant hydrolase enzymes. Such solutions in principle have any physical form, e.g., tablets, solutions, cell cultures, etc. For example, cells transformed with the recombinant genes for triazine hydrolases provided herein are used to express the hydrolases. The cells are mixed with soil or water for decontamination where the expressed enzyme degrades or hydrolyzes the triazine derivative, thus purifying the soil or water sample.

Integrated Systems

The present invention provides computers, computer readable media and integrated systems comprising character strings corresponding to the sequence information herein for the polypeptides and nucleic acids herein, including, e.g., those sequences listed herein and the various silent substitutions and conservative substitutions thereof.

Various methods and genetic algorithms (GOs) known in the art can be used to detect homology or similarity between different character strings, or can be used to perform other desirable functions such as to control output files, provide the basis for making presentations of information including the sequences and the like. Examples include BLAST, discussed supra.

Thus, different types of homology and similarity of various stringency and length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among 4 principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). An example of a software package with GOs for calculating sequence similarity is BLAST, which can be adapted to the present invention by inputting character strings corresponding to the sequences herein.

Similarly, standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting a character string corresponding to the triazine hydrolases of the invention (either nucleic acids or proteins, or both). For example, the integrated systems can include the foregoing software having the appropriate character string information, e.g., used in conjunction with a user interface (e.g., a GUI in a standard operating system such as a Windows, Macintosh or LINUX system) to manipulate strings of characters. As noted, specialized alignment programs such as BLAST can also be incorporated into the systems of the invention for alignment of nucleic acids or proteins (or corresponding character strings).

Integrated systems for analysis in the present invention typically include a digital computer with GO software for aligning sequences, as well as data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visualbasic, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation.

The software can also include output elements for controlling nucleic acid synthesis (e.g., based upon a sequence or an alignment of a sequences herein) or other operations which occur downstream from an alignment or other operation performed using a character string corresponding to a sequence herein.

In an additional aspect, the present invention provides kits embodying the methods, composition, systems and apparatus herein. Kits of the invention optionally comprise one or more of the following: (1) an apparatus, system, system component or apparatus component as described herein; (2) instructions for practicing the methods described herein, and/or for operating the apparatus or apparatus components herein and/or for using the compositions herein; (3) one or more triazine hydrolase composition or component; (4) a container for holding components or compositions, and, (5) packaging materials.

In a further aspect, the present invention provides for the use of any apparatus, apparatus component, composition or kit herein, for the practice of any method or assay herein, and/or for the use of any apparatus or kit to practice any assay or method herein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

### Table 3

Product Formation in nM/h Using Following Reaction Conditions:

20 ul washed and induced cells OD600=3 in a total volume of 100 μl 10 mM

NH₃Acetate pH 6.8, 250 μM substrate, incubation at 22° C.

TABLE 3
Product formation in nM/h using following reaction conditions:
20 ul washed and induced cells OD600 = in a total volume of
100 μl 10 nM
NH₃Acetate pH 6.8, 250 μM substrate, incubation at 22° C.

| | substrate | Atrazine | Atratone | Ametryn | NMeAtrazine | Aminoatrazine |
|---|---|---|---|---|---|---|
| nc | | 0 | 0 | 0 | 0 | 0 |
| atzA | | 58899 | 0 | 0 | 0 | 0 |
| Hit 1 | 1D.7 G8 | 13800 | 12364 | 1717 | 2198 | 190877 |
| Hit 2 | 1U.3 A7 | 16381 | 14586 | 2489 | 3830 | 262994 |
| Hit 3 | 1D.1 D2 | 21585 | 23685 | 667 | 762 | 118358 |
| Hit 4 | 1D.1 C10 | 20654 | 18738 | 420 | 1273 | 224186 |
| Hit 5 | 1D.10 A7 | 15715 | 12744 | 2035 | 1755 | 136259 |
| Hit 6 | 1U.3 F12 | 8639 | 7752 | 638 | 2080 | 143177 |
| Hit 7 | 1U.5 H8 | 4474 | 4685 | 348 | 2106 | 88757 |
| Hit 8 | 1D.1 C3 | 80746 | 0 | 0 | 0 | 0 |
| Hit 9 | 1D.5 H6 | 29313 | 0 | 0 | 0 | 0 |

### Table 4

Product Formation in nM/h Using Following Reaction Conditions:

20 ul washed and induced cells OD600=3 in a total volume of 100 μl 10 mM

NH₃Acetate pH 6.8, 250 μM substrate, incubation at 37° C.

Product formation in nM/h using following reaction conditions:
20 ul washed and induced cells OD600 = 3 in a total volume of
100 μl 10 mM
NH₃Acetate pH 6.8, 250 μM substrate, incubation at 37° C.

| | substrate | Propazine | Prometon | Prometryn | NMePropazine | Aminopropazine |
|---|---|---|---|---|---|---|
| nc | | 0 | 0 | 0 | 0 | 0 |
| atzA | | 26 | 0 | 0 | 0 | 0 |
| Hit 1 | 1D.7 G8 | 0 | 434 | 0 | 0 | 33355 |
| Hit 2 | 1U.3 A7 | 0 | 892 | 0 | 23 | 46018 |
| Hit 3 | 1D.1 D2 | 0 | 49 | 0 | 0 | 1312 |
| Hit 4 | 1D.1 C10 | 0 | 746 | 16 | 19 | 11322 |
| Hit 5 | 1D.10 A7 | 0 | 818 | 0 | 18 | 13951 |
| Hit 6 | 1U.3 F12 | 56 | 2888 | 301 | 1453 | 20856 |
| Hit 7 | 1U.5 H8 | 0 | 1423 | 27 | 180 | 29753 |
| Hit 8 | 1D.1 C3 | 16 | 0 | 0 | 0 | 0 |
| Hit 9 | 1D.5 H6 | 72 | 0 | 0 | 0 | 0 |

TABLE 5

| | LIBRARY | | | | | | | | | | |
|---|---|---|---|---|---|---|---|---|---|---|---|
| | MEMBER | | | | | | | | | | Substrate |
| SEQ. ID NO. | NO. | 84 | 92 | 125 | 217 | 219 | 253 | 255 | 328 | 331 | Activity |
| Seq ID NO 97 | 1 | F | L | D | I | P | I | G | D | C | |
| Seq ID NO 98 | 2 | F | L | D | I | P | I | G | D | S | |
| Seq ID NO 99 | 3 | F | L | D | I | P | I | G | N | C | |
| Seq ID NO. 100 | 4 | F | L | D | I | P | I | G | N | S | |
| Seq ID NO 101 | 5 | F | L | D | I | P | I | W | D | C | |
| Seq ID NO 102 | 6 | F | L | D | I | P | I | W | D | S | |
| Seq ID NO 103 | 7 | F | L | D | I | P | I | W | N | C | |
| Seq ID NO 104 | 8 | F | L | D | I | P | I | W | N | S | |
| Seq ID NO 105 | 9 | F | L | D | I | P | L | G | D | C | |
| Seq ID NO 106 | 10 | F | L | D | I | P | L | G | D | S | |
| Seq ID NO 107 | 11 | F | L | D | I | P | L | G | N | C | |
| Seq IO NO 108 | 12 | F | L | D | I | P | L | G | N | S | |
| Seq ID NO 109 | 13 | F | L | D | I | P | L | W | D | C | |
| Seq ID NO 110 | 14 | F | L | D | I | P | L | W | D | S | |
| Seq ID NO 111 | 15 | F | L | D | I | P | L | W | N | C | |
| Seq ID NO 112 | 16 | F | L | D | I | P | L | W | N | S | |
| Seq ID NO 113 | 17 | F | L | D | I | T | I | G | D | C | |
| Seq ID NO 114 | 18 | F | L | D | I | T | I | G | D | S | |
| Seq ID NO 115 | 19 | F | L | D | I | T | I | G | N | C | |
| Seq ID NO 116 | 20 | F | L | D | I | T | I | G | N | S | |
| Seq ID NO 117 | 21 | F | L | D | I | T | I | W | D | C | |
| Seq ID NO 118 | 22 | F | L | D | I | T | I | W | D | S | |
| Seq ID NO 119 | 23 | F | L | D | I | T | I | W | N | C | |
| Seq ID NO 120 | 24 | F | L | D | I | T | I | W | N | S | |
| Seq ID NO 121 | 25 | F | L | D | I | T | L | G | D | C | |
| Seq ID NO 122 | 26 | F | L | D | I | T | L | G | D | S | |
| Seq ID NO 123 | 27 | F | L | D | I | T | L | G | N | C | |
| Seq ID NO 124 | 28 | F | L | D | I | T | L | G | N | S | |
| Seq ID NO 125 | 29 | F | L | D | I | T | L | W | D | C | |
| Seq ID NO 126 | 30 | F | L | D | I | T | L | W | D | S | |
| Seq ID NO 127 | 31 | F | L | D | I | T | L | W | N | C | |
| Seq ID NO. 128 | 32 | F | L | D | I | T | L | W | N | S | |
| Seq ID NO. 129 | 33 | F | L | D | T | P | I | G | D | C | |
| Seq ID NO 130 | 34 | F | L | D | T | P | I | G | D | S | |
| Seq ID NO 131 | 35 | F | L | D | T | P | I | G | N | C | |
| Seq ID NO 132 | 36 | F | L | D | T | P | I | G | N | S | |
| Seq ID NO 133 | 37 | F | L | D | T | P | I | W | D | C | |
| Seq ID NO 134 | 38 | F | L | D | T | P | I | W | D | S | |
| Seq ID NO 135 | 39 | F | L | D | T | P | I | W | N | C | |
| Seq ID NO 136 | 40 | F | L | D | T | P | I | W | N | S | |
| Seq ID NO 137 | 41 | F | L | D | T | P | L | G | D | C | |
| Seq ID NO 138 | 42 | F | L | D | T | P | L | G | D | S | |
| Seq ID NO. 139 | 43 | F | L | D | T | P | L | G | N | C | |
| Seq ID NO 140 | 44 | F | L | D | T | P | L | G | N | S | |
| Seq ID NO 141 | 45 | F | L | D | T | P | L | W | D | C | |
| Seq ID NO 142 | 46 | F | L | D | T | P | L | W | D | S | |
| Seq ID NO 143 | 47 | F | L | D | T | P | L | W | N | C | |
| Seq ID NO 144 | 48 | F | L | D | T | P | L | W | N | S | |
| Seq ID NO 145 | 49 | F | L | D | T | T | I | G | D | C | atratone |
| Seq ID NO 146 | 50 | F | L | D | T | T | I | G | D | S | |
| Seq ID NO 147 | 51 | F | L | D | T | T | I | G | N | C | |
| Seq ID NO 148 | 52 | F | L | D | T | T | I | G | N | S | |
| Seq ID NO 149 | 53 | F | L | D | T | T | I | W | D | C | atratone |
| Seq ID NO 150 | 54 | F | L | D | T | T | I | W | D | S | |
| Seq ID NO 151 | 55 | F | L | D | T | T | I | W | N | C | |
| Seq ID NO 152 | 56 | F | L | D | T | T | I | W | N | S | |
| Seq ID NO 153 | 57 | F | L | D | T | T | L | G | D | C | atratone |
| Seq ID NO 154 | 58 | F | L | D | T | T | L | G | D | S | |
| Seq ID NO 155 | 59 | F | L | D | T | T | L | G | N | C | |
| Seq ID NO 156 | 60 | F | L | D | T | T | L | G | N | S | |
| Seq ID NO 157 | 61 | F | L | D | T | T | L | W | D | C | atratone |
| Seq ID NO 158 | 62 | F | L | D | T | T | L | W | D | S | |
| Seq ID NO 159 | 63 | F | L | D | T | T | L | W | N | C | |
| Seq ID NO 160 | 64 | F | L | D | T | T | L | W | N | S | |
| Seq ID NO 161 | 65 | F | L | E | I | P | I | G | D | C | |
| Seq ID NO. 162 | 66 | F | L | E | I | P | I | G | D | S | |
| Seq ID NO. 163 | 67 | F | L | E | I | P | I | G | N | C | |
| Seq ID NO 164 | 68 | F | L | E | I | P | I | G | N | S | |
| Seq ID NO 165 | 69 | F | L | E | I | P | I | W | D | C | |
| Seq ID NO 166 | 70 | F | L | E | I | P | I | W | D | S | |
| Seq ID NO 167 | 71 | F | L | E | I | P | I | W | N | C | |
| Seq ID NO 168 | 72 | F | L | E | I | P | I | W | N | S | |
| Seq ID NO 169 | 73 | F | L | E | I | P | L | G | D | C | |
| Seq ID NO. 170 | 74 | F | L | E | I | P | L | G | D | S | |
| Seq ID NO 171 | 75 | F | L | E | I | P | L | G | N | C | |
| Seq ID NO 172 | 76 | F | L | E | I | P | L | G | N | S | |
| Seq ID NO 173 | 77 | F | L | E | I | P | L | W | D | C | |
| Seq ID NO 174 | 78 | F | L | E | I | P | L | w | D | S | |
| Seq ID NO 175 | 79 | F | L | E | I | P | L | W | N | C | |
| Seq ID NO 176 | 80 | F | L | E | I | P | L | W | N | S | |
| Seq ID NO 177 | 81 | F | L | E | I | T | I | G | D | C | |
| Seq ID NO 178 | 82 | F | L | E | I | T | I | G | D | S | |
| Seq ID NO 179 | 83 | F | L | E | I | T | I | G | N | C | |
| Seq ID NO 180 | 84 | F | L | E | I | T | I | G | N | S | |
| Seq ID NO 181 | 85 | F | L | E | I | T | I | W | D | C | |
| Seq ID NO 182 | 86 | F | L | E | I | T | I | W | D | S | |
| Seq ID NO 183 | 87 | F | L | E | I | T | I | W | N | C | |
| Seq ID NO 184 | 88 | F | L | E | I | T | I | W | N | S | |
| Seq ID NO 185 | 89 | F | L | E | I | T | L | G | D | C | |
| Seq ID NO 186 | 90 | F | L | E | I | T | L | G | D | S | |
| Seq ID NO 187 | 91 | F | L | E | I | T | L | G | N | C | |
| Seq ID NO 188 | 92 | F | L | E | I | T | L | G | N | S | |
| Seq ID NO 189 | 93 | F | L | E | I | T | L | W | D | C | |
| Seq ID NO 190 | 94 | F | L | E | I | T | L | W | D | S | |
| Seq ID NO 191 | 95 | F | L | E | I | T | L | W | N | C | |
| Seq ID NO 192 | 96 | F | L | E | I | T | L | W | N | S | |
| Seq ID NO 193 | 97 | F | L | E | T | P | I | G | D | C | |
| Seq ID NO 194 | 98 | F | L | E | T | P | I | G | D | S | |
| Seq ID NO 195 | 99 | F | L | E | T | P | I | G | N | C | |
| Seq ID NO 196 | 100 | F | L | E | T | P | I | G | N | S | |
| Seq ID NO 197 | 101 | F | L | E | T | P | I | W | D | C | |
| Seq ID NO 198 | 102 | F | L | E | T | P | I | W | D | S | |
| Seq ID NO 199 | 103 | F | L | E | T | P | I | W | N | C | |
| Seq ID NO 200 | 104 | F | L | E | T | P | I | W | N | S | |
| Seq ID NO 201 | 105 | F | L | E | T | P | L | G | D | C | |
| Seq ID NO 202 | 106 | F | L | E | T | P | L | G | D | S | |
| Seq ID NO 203 | 107 | F | L | E | T | P | L | G | N | C | |
| Seq ID NO 204 | 108 | F | L | E | T | P | L | G | N | S | |
| Seq ID NO 205 | 109 | F | L | E | T | P | L | W | D | C | |
| Seq ID NO 206 | 110 | F | L | E | T | P | L | W | D | S | |
| Seq ID NO 207 | 111 | F | L | E | T | P | L | W | N | C | |
| Seq ID NO 208 | 112 | F | L | E | T | P | L | W | N | S | |
| Seq ID NO 209 | 113 | F | L | E | T | T | I | G | D | C | atratone |
| Seq ID NO 210 | 114 | F | L | E | T | T | I | G | D | S | |
| Seq ID NO 211 | 115 | F | L | E | T | T | I | G | N | C | |
| Seq ID NO 212 | 116 | F | L | E | T | T | I | G | N | S | |
| Seq ID NO 213 | 117 | F | L | E | T | T | I | W | D | C | atratone |
| Seq ID NO 214 | 118 | F | L | E | T | T | I | W | D | S | |
| Seq ID NO 215 | 119 | F | L | E | T | T | I | W | N | C | |
| Seq ID NO 216 | 120 | F | L | E | T | T | I | W | N | S | |
| Seq ID NO 217 | 121 | F | L | E | T | T | L | G | D | C | nme-atrazine, |
| | | | | | | | | | | | ametryn, |
| | | | | | | | | | | | atratone |
| Seq ID NO 218 | 122 | F | L | E | T | T | L | G | D | S | |
| Seq ID NO. 219 | 123 | F | L | E | T | T | L | G | N | C | |
| Seq ID NO 220 | 124 | F | L | E | T | T | L | G | N | S | atrazine |
| Seq ID NO 221 | 125 | F | L | E | T | T | L | W | D | C | nme-atrazine, |
| | | | | | | | | | | | ametryn, |
| | | | | | | | | | | | atratone |
| Seq ID NO. 222 | 126 | F | L | E | T | T | L | W | D | S | |
| Seq ID NO 223 | 127 | F | L | E | T | T | L | W | N | C | |
| Seq ID NO. 224 | 128 | F | L | E | T | T | L | W | N | S | atrazine |
| Seq ID NO 225 | 129 | F | V | D | I | P | I | G | D | C | |
| Seq ID NO 226 | 130 | F | V | D | I | P | I | G | D | S | |
| Seq ID NO 227 | 131 | F | V | D | I | P | I | G | N | C | |
| Seq ID NO 228 | 132 | F | V | D | I | P | I | G | N | S | |
| Seq ID NO 229 | 133 | F | V | D | I | P | I | W | D | C | |
| Seq ID NO 230 | 134 | F | V | D | I | P | I | W | D | S | |
| Seq ID NO 231 | 135 | F | V | D | I | P | I | W | N | C | |
| Seq ID NO 232 | 136 | F | V | D | I | P | I | W | N | S | |
| Seq ID NO 233 | 137 | F | V | D | I | P | L | G | D | C | |
| Seq. ID NO 234 | 138 | F | V | D | I | P | L | G | D | S | |
| Seq ID NO 235 | 139 | F | V | D | I | P | L | G | N | C | |
| Seq ID NO 236 | 140 | F | V | D | I | P | L | G | N | S | |
| Seq ID NO 237 | 141 | F | V | D | I | P | L | W | D | C | |
| Seq ID NO 238 | 142 | F | V | D | I | p | L | W | D | S | |
| Seq ID NO 239 | 143 | F | V | D | I | P | L | W | N | C | |
| Seq ID NO 240 | 144 | F | V | D | I | P | L | W | N | S | |
| Seq ID NO 241 | 145 | F | V | D | I | T | I | G | D | C | |
| Seq ID NO 242 | 146 | F | V | D | I | T | I | G | D | S | |
| Seq ID NO 243 | 147 | F | V | D | I | T | I | G | N | C | |
| Seq ID NO 244 | 148 | F | V | D | I | T | I | G | N | S | |
| Seq ID NO. 245 | 149 | F | V | D | I | T | I | W | D | C | |
| Seq ID NO. 246 | 150 | F | V | D | I | T | I | W | D | S | |
| Seq. ID NO 247 | 151 | F | V | D | I | T | I | W | N | C | |
| Seq ID NO 248 | 152 | F | V | D | I | T | I | W | N | S | |
| Seq ID NO 249 | 153 | F | V | D | I | T | L | G | D | C | |
| Seq ID NO 250 | 154 | F | V | D | I | T | L | G | D | S | |
| Seq ID NO 251 | 155 | F | V | D | I | T | L | G | N | C | |
| Seq ID NO 252 | 156 | F | V | D | I | T | L | G | N | S | |
| Seq ID NO 253 | 157 | F | V | D | I | T | L | W | D | C | |
| Seq ID NO 254 | 158 | F | V | D | I | T | L | W | D | S | |
| Seq ID NO 255 | 159 | F | V | D | I | T | L | W | N | C | |
| Seq ID NO. 256 | 160 | F | V | D | I | T | L | W | N | S | |
| Seq ID NO 257 | 161 | F | V | D | T | P | I | G | D | C | |
| Seq ID NO 258 | 162 | F | V | D | T | P | I | G | D | S | |
| Seq ID NO 259 | 163 | F | V | D | T | P | I | G | N | C | |
| Seq ID NO 260 | 164 | F | V | D | T | P | I | G | N | S | |
| Seq ID NO 261 | 165 | F | V | D | T | P | I | W | D | C | |
| Seq ID NO 262 | 166 | F | V | D | T | P | I | W | D | S | |
| Seq ID NO 263 | 167 | F | V | D | T | P | I | W | N | C | |
| Seq ID NO 264 | 168 | F | V | D | T | P | L | W | N | S | |
| Seq ID NO 265 | 169 | F | V | D | T | P | L | G | D | C | |
| Seq ID NO 266 | 170 | F | V | D | T | P | L | G | D | S | |
| Seq ID NO 267 | 171 | F | V | D | T | P | L | G | N | C | |
| Seq ID NO 268 | 172 | F | V | D | T | P | L | G | N | S | |
| Seq ID NO 269 | 173 | F | V | D | T | P | L | W | D | C | |
| Seq ID NO 270 | 174 | F | V | D | T | P | L | W | D | S | |
| Seq. ID NO 271 | 175 | F | V | D | T | P | L | W | N | C | |
| Seq ID NO 272 | 176 | F | V | D | T | P | L | W | N | S | |
| Seq ID NO. 273 | 177 | F | V | D | T | T | I | G | D | C | |
| Seq ID NO 274 | 178 | F | V | D | T | T | I | G | D | S | |
| Seq ID NO 275 | 179 | F | V | D | T | T | I | G | N | C | |
| Seq ID NO 276 | 180 | F | V | D | T | T | I | G | N | S | |
| Seq ID NO. 277 | 181 | F | V | D | T | T | I | W | D | C | |
| Seq ID NO 278 | 182 | F | V | D | T | T | I | W | D | S | |
| Seq ID NO 279 | 183 | F | V | D | T | T | I | W | N | C | |
| Seq ID NO 280 | 184 | F | V | D | T | T | I | W | N | S | |
| Seq ID NO 281 | 185 | F | V | D | T | T | L | G | D | C | |
| Seq ID NO 282 | 186 | F | V | D | T | T | L | G | D | S | |
| Seq ID NO 283 | 187 | F | V | D | T | T | L | G | N | C | |
| Seq ID NO 284 | 188 | F | V | D | T | T | L | G | N | S | |
| Seq ID NO 285 | 189 | F | V | D | T | T | L | W | D | C | |
| Seq ID NO 286 | 190 | F | V | D | T | T | L | W | D | S | |
| Seq ID NO 287 | 191 | F | V | D | T | T | L | W | N | C | |
| Seq. ID NO 288 | 192 | F | V | D | T | T | L | W | N | S | |
| Seq ID NO. 289 | 193 | F | V | E | I | P | I | G | D | C | |
| Seq ID NO 290 | 194 | F | V | E | I | P | I | G | D | S | |
| Seq ID NO 291 | 195 | F | V | E | I | P | I | G | N | C | |
| Seq ID NO 292 | 196 | F | V | E | I | P | I | G | N | S | |
| Seq ID NO 293 | 197 | F | V | E | I | P | I | W | D | C | |
| Seq ID NO 294 | 198 | F | V | E | I | P | I | W | D | S | |
| Seq ID NO 295 | 199 | F | V | E | I | P | I | W | N | C | |
| Seq ID NO 296 | 200 | F | V | E | I | P | I | W | N | S | |
| Seq ID NO 297 | 201 | F | V | E | I | P | L | G | D | C | |
| Seq ID NO 298 | 202 | F | V | E | I | P | L | G | D | S | |
| Seq ID NO 299 | 203 | F | V | E | I | P | L | G | N | C | |
| Seq ID NO 300 | 204 | F | V | E | I | P | L | G | N | S | |
| Seq ID NO 301 | 205 | F | V | E | I | P | L | W | D | C | |
| Seq ID NO 302 | 206 | F | V | E | I | P | L | W | D | S | |
| Seq ID NO 303 | 207 | F | V | E | I | P | L | W | N | C | |
| Seq ID NO. 304 | 208 | F | V | E | I | P | L | W | N | S | |
| Seq ID NO 305 | 209 | F | V | E | I | T | I | G | D | C | |
| Seq ID NO. 306 | 210 | F | V | E | I | T | I | G | D | S | |
| Seq ID NO 307 | 211 | F | V | E | I | T | I | G | N | C | |
| Seq ID NO 308 | 212 | F | V | E | I | T | I | G | N | S | |
| Seq ID NO 309 | 213 | F | V | E | I | T | I | W | D | C | |
| Seq ID NO 310 | 214 | F | V | E | I | T | I | W | D | S | |
| Seq ID NO 311 | 215 | F | V | E | I | T | I | W | N | C | |
| Seq ID NO 312 | 216 | F | V | E | I | T | I | W | N | S | |
| Seq ID NO 313 | 217 | F | V | E | I | T | L | G | D | C | |
| Seq ID NO 314 | 218 | F | V | E | I | T | L | G | D | S | |
| Seq ID NO 315 | 219 | F | V | E | I | T | L | G | N | C | |
| Seq ID NO 316 | 220 | F | V | E | I | T | L | G | N | S | |
| Seq ID NO 317 | 221 | F | V | E | I | T | L | W | D | C | |
| Seq ID NO 318 | 222 | F | V | E | I | T | L | W | D | S | |
| Seq ID NO 319 | 223 | F | V | E | I | T | L | W | N | C | |
| Seq ID NO 320 | 224 | F | V | E | I | T | L | W | N | S | |
| Seq ID NO 321 | 225 | F | V | E | T | P | I | G | D | C | |
| Seq ID NO 322 | 226 | F | V | E | T | P | I | G | D | S | |
| Seq ID NO. 323 | 227 | F | V | E | T | P | I | G | N | C | |
| Seq ID NO 324 | 228 | F | V | E | T | F | I | G | N | S | |
| Seq ID NO 325 | 229 | F | V | E | T | P | I | W | D | C | |
| Seq ID NO 326 | 230 | F | V | E | T | P | I | W | D | S | |
| Seq ID NO 327 | 231 | F | V | E | T | P | I | W | N | C | |
| Seq ID NO 328 | 232 | F | V | E | T | P | I | W | N | S | |
| Seq ID NO 329 | 233 | F | V | E | T | P | L | G | D | C | |
| Seq. ID NO 330 | 234 | F | V | E | T | P | L | G | D | S | |
| Seq ID NO 331 | 235 | F | V | E | T | P | L | G | N | C | |
| Seq ID NO. 332 | 236 | F | V | E | T | P | L | G | N | S | |
| Seq ID NO 333 | 237 | F | V | E | T | P | L | W | D | C | |
| Seq ID NO 334 | 238 | F | V | E | T | P | L | W | D | S | |
| Seq ID NO 335 | 239 | F | V | E | T | P | L | W | N | C | |
| Seq ID NO 336 | 240 | F | V | E | T | P | L | W | N | S | |
| Seq ID NO 337 | 241 | F | V | E | T | T | I | G | D | C | |
| Seq ID NO 338 | 242 | F | V | E | T | T | I | G | D | S | |
| Seq ID NO 339 | 243 | F | V | F | T | T | I | G | N | C | |
| Seq ID NO 340 | 244 | F | V | E | T | T | I | G | N | S | propazine |
| Seq ID NO 341 | 245 | F | V | E | T | T | I | W | D | C | |
| Seq ID NO 342 | 246 | F | V | E | T | T | I | W | D | S | |
| Seq ID NO 343 | 247 | F | V | E | T | T | I | W | N | C | |
| Seq ID NO 344 | 248 | F | V | E | T | T | I | W | N | S | propazine |
| Seq ID NO 345 | 249 | F | V | E | T | T | L | G | D | C | amino- |
| | | | | | | | | | | | propazine |
| Seq ID NO 346 | 250 | F | V | E | T | T | L | G | D | S | |
| Seq ID NO 347 | 251 | F | V | E | T | T | L | G | N | C | |
| Seq ID NO 348 | 252 | F | V | E | T | T | L | G | N | S | propazine, |
| | | | | | | | | | | | atrazine |
| Seq ID NO 349 | 253 | F | V | E | T | T | L | W | D | C | |
| Seq ID NO 350 | 254 | F | V | E | T | T | L | W | D | S | |
| Seq ID NO 351 | 255 | F | V | E | T | T | L | W | N | C | |
| Seq ID NO 352 | 256 | F | V | E | T | T | L | W | N | S | propazine, |
| | | | | | | | | | | | atrazine |
| Seq ID NO 353 | 257 | L | L | D | I | P | I | G | D | C | |
| Seq ID NO 354 | 258 | L | L | D | I | P | I | G | D | S | |
| Seq ID NO 355 | 259 | L | L | D | I | P | I | G | N | C | |
| Seq ID NO 356 | 260 | L | L | D | I | P | I | G | N | S | |
| Seq ID NO 357 | 261 | L | L | D | I | P | I | W | D | C | |
| Seq ID NO 358 | 262 | L | L | D | I | P | I | W | D | S | |
| Seq ID NO 359 | 263 | L | L | D | I | P | I | W | N | C | |
| Seq ID NO 360 | 264 | L | L | D | I | P | I | W | N | S | |
| Seq ID NO 361 | 265 | L | L | D | I | P | L | G | D | C | |
| Seq ID NO 362 | 266 | L | L | 0 | I | P | L | G | D | S | |
| Seq ID NO 363 | 267 | L | L | D | I | P | L | G | N | C | |
| Seq ID NO 364 | 268 | L | L | D | I | P | L | G | N | S | |
| Seq ID NO 365 | 269 | L | L | D | I | P | L | W | D | C | |
| Seq ID NO 366 | 270 | L | L | D | I | P | L | W | D | S | |
| Seq ID NO 367 | 271 | L | L | D | I | P | L | W | N | C | |
| Seq ID NO 368 | 272 | L | L | D | I | P | L | W | N | S | |
| Seq ID NO. 369 | 273 | L | L | D | I | T | I | G | D | C | |
| Seq ID NO 370 | 274 | L | L | D | I | T | I | G | D | S | |
| Seq ID NO 371 | 275 | L | L | D | I | T | I | G | N | C | |
| Seq ID NO 372 | 276 | L | L | D | I | T | I | G | N | S | |
| Seq ID NO 373 | 277 | L | L | D | I | T | I | W | D | C | |
| Seq ID NO 374 | 278 | L | L | D | I | T | I | W | D | S | |
| Seq ID NO 375 | 279 | L | L | D | I | T | I | W | N | C | |
| Seq ID NO 376 | 280 | L | L | D | I | T | I | W | N | S | |
| Seq ID NO 377 | 281 | L | L | D | I | T | L | G | D | C | |
| Seq ID NO 378 | 282 | L | L | D | I | T | L | G | D | S | |
| Seq ID NO 379 | 283 | L | L | D | I | T | L | G | N | C | |
| Seq ID NO 380 | 284 | L | L | D | I | T | L | G | N | S | |
| Seq ID NO 381 | 285 | L | L | D | I | T | L | W | D | C | |
| Seq ID NO 382 | 286 | L | L | D | I | T | L | W | D | S | |
| Seq ID NO 383 | 287 | L | L | D | I | T | L | W | N | C | |
| Seq ID NO 384 | 288 | L | L | D | I | T | L | W | N | S | |
| Seq ID NO 385 | 289 | L | L | D | T | P | I | G | D | C | |
| Seq ID NO 386 | 290 | L | L | D | T | P | I | G | D | 5 | |
| Seq ID NO 387 | 291 | L | L | D | T | P | I | 0 | N | C | |
| Seq ID NO 388 | 292 | L | L | D | T | P | I | G | N | S | |
| Seq ID NO 389 | 293 | L | L | D | T | P | I | W | D | C | |
| Seq ID NO 390 | 294 | L | L | D | T | P | I | W | D | S | |
| Seq ID NO 391 | 295 | L | L | D | T | P | I | W | N | C | |
| Seq ID NO 392 | 296 | L | L | D | T | P | I | W | N | S | |
| Seq ID NO 393 | 297 | L | L | D | T | P | L | G | D | C | |
| Seq ID NO 394 | 298 | L | L | D | T | P | L | G | D | S | |
| Seq ID NO 395 | 299 | L | L | D | T | P | L | G | N | C | |
| Seq ID NO 396 | 300 | L | L | D | T | P | L | G | N | S | |
| Seq ID NO 397 | 301 | L | L | D | T | P | L | W | D | C | |
| Seq ID NO 398 | 302 | L | L | D | T | P | L | W | D | S | |
| Seq ID NO 399 | 303 | L | L | D | T | P | L | W | N | C | |
| Seq ID NO 400 | 304 | L | L | D | T | P | L | W | N | S | |
| Seq ID NO 401 | 305 | L | L | D | T | T | I | G | D | C | amino- |
| | | | | | | | | | | | atrazine, |
| | | | | | | | | | | | atratone |
| Seq ID NO 402 | 306 | L | L | D | T | T | I | G | D | S | |
| Seq ID NO 403 | 307 | L | L | D | T | T | I | G | N | C | |
| Seq ID NO 404 | 308 | L | L | D | T | T | I | G | N | S | |
| Seq ID NO 405 | 309 | L | L | D | T | T | I | W | D | C | amino- |
| | | | | | | | | | | | atrazine, |
| | | | | | | | | | | | atratone |
| Seq ID NO 406 | 310 | L | L | D | T | T | I | W | D | S | |
| Seq ID NO 407 | 311 | L | L | D | T | T | I | W | N | C | |
| Seq ID NO 408 | 312 | L | L | D | T | T | I | W | N | S | |
| Seq ID NO 409 | 313 | L | L | D | T | T | L | G | D | C | nme-atrazine, |
| | | | | | | | | | | | amino- |
| | | | | | | | | | | | atrazine, |
| | | | | | | | | | | | ametryn, |
| | | | | | | | | | | | atratone |
| Seq ID NO 410 | 314 | L | L | D | T | T | L | G | D | S | ametryn |
| Seq ID NO 411 | 315 | L | L | D | T | T | L | G | N | C | |
| Seq ID NO 412 | 316 | L | L | D | T | T | L | G | N | S | |
| Seq ID NO 413 | 317 | L | L | D | T | T | L | W | D | C | nme-atrazine, |
| | | | | | | | | | | | amino- |
| | | | | | | | | | | | atrazine, |
| | | | | | | | | | | | ametryn, |
| | | | | | | | | | | | atratone |
| Seq ID NO 414 | 318 | L | L | D | T | T | L | W | G | S | ametryn |
| Seq ID NO 415 | 319 | L | L | D | T | T | L | W | N | C | |
| Seq ID NO. 416 | 320 | L | L | D | T | T | L | W | N | S | |
| Seq ID NO 417 | 321 | L | L | E | I | P | I | G | G | C | |
| Seq ID NO 418 | 322 | L | L | E | I | P | I | G | G | S | |
| Seq ID NO 419 | 323 | L | L | E | I | P | I | G | N | C | |
| Seq ID NO 420 | 324 | L | L | E | I | P | I | G | N | S | |
| Seq ID NO 421 | 325 | L | L | E | I | P | I | W | G | C | |
| Seq ID NO 422 | 326 | L | L | E | I | P | I | W | G | S | |
| Seq ID NO 423 | 327 | L | L | E | I | P | I | W | N | C | |
| Seq ID NO 424 | 328 | L | L | E | I | P | I | W | N | S | |
| Seq ID NO 425 | 329 | L | L | E | I | P | L | G | G | C | |
| Seq ID NO 426 | 330 | L | L | E | I | P | L | G | G | S | |
| Seq ID NO 427 | 331 | L | L | E | I | P | L | G | N | C | |
| Seq ID NO 428 | 332 | L | L | E | I | P | L | G | N | S | |
| Seq ID NO 429 | 333 | L | L | E | I | P | L | W | G | C | |
| Seq ID NO 430 | 334 | L | L | E | I | F | L | W | G | S | |
| Seq ID NO 431 | 335 | L | L | E | I | P | L | W | N | C | |
| Seq ID NO 432 | 336 | L | L | E | I | P | L | W | N | S | |
| Seq ID NO 433 | 337 | L | L | E | I | T | I | G | G | C | |
| Seq ID NO 434 | 338 | L | L | E | I | T | I | G | G | S | |
| Seq ID NO 435 | 339 | L | L | E | I | T | I | G | N | C | |
| Seq ID NO 436 | 340 | L | L | E | I | T | I | G | N | S | |
| Seq ID NO 437 | 341 | L | L | E | I | T | I | W | G | C | |
| Seq ID NO 438 | 342 | L | L | E | I | T | I | W | G | S | |
| Seq ID NO 439 | 343 | L | L | E | I | T | I | W | N | C | |
| Seq ID NO 440 | 344 | L | L | E | I | T | I | W | N | S | |
| Seq ID NO 441 | 345 | L | L | E | I | T | L | G | G | C | nme-atrazine |
| Seq ID NO 442 | 346 | L | L | E | I | T | L | G | G | S | |
| Seq ID NO 443 | 347 | L | L | E | I | T | L | G | N | C | |
| Seq ID NO 444 | 348 | L | L | E | I | T | L | G | N | S | |
| Seq ID NO 445 | 349 | L | L | E | I | T | L | W | D | C | nme-atrazine |
| Seq ID NO 446 | 350 | L | L | E | I | T | L | W | D | S | |
| Seq ID NO 447 | 351 | L | L | E | I | T | L | W | N | C | |
| Seq ID NO 448 | 352 | L | L | E | I | T | L | W | N | S | |
| Seq ID NO 449 | 353 | L | L | E | T | P | I | G | D | C | |
| Seq ID NO 450 | 354 | L | L | E | T | P | I | G | D | S | |
| Seq ID NO 451 | 355 | L | L | E | T | P | I | G | N | C | |
| Seq ID NO 452 | 356 | L | L | E | T | P | I | G | N | S | |
| Seq ID NO 453 | 357 | L | L | E | T | P | I | W | D | C | |
| Seq ID NO 454 | 358 | L | L | E | T | P | I | W | D | S | |
| Seq ID NO 455 | 359 | L | L | E | T | P | I | W | N | C | |
| Seq ID NO 456 | 360 | L | L | E | T | P | I | W | N | S | |
| Seq ID NO 457 | 361 | L | L | E | T | P | L | G | D | C | |
| Seq ID NO 458 | 362 | L | L | E | T | P | L | G | D | S | |
| Seq ID NO 459 | 363 | L | L | E | T | P | L | G | N | C | |
| Seq ID NO 460 | 364 | L | L | E | T | P | L | G | N | S | |
| Seq ID NO 461 | 365 | L | L | E | T | P | L | W | D | C | |
| Seq ID NO 462 | 366 | L | L | E | T | P | L | W | D | S | |
| Seq ID NO 463 | 367 | L | L | E | T | P | L | W | N | C | |
| Seq ID NO 464 | 368 | L | L | E | T | P | L | W | N | S | |
| Seq ID NO 465 | 369 | L | L | E | T | T | I | G | D | C | nme-atrazine, |
| | | | | | | | | | | | amino- |
| | | | | | | | | | | | atrazine, |
| | | | | | | | | | | | ametryn, |
| | | | | | | | | | | | atratone |
| Seq ID NO 466 | 370 | L | L | E | T | T | I | G | D | S | ametryn |
| Seq ID NO 467 | 371 | L | L | E | T | T | I | G | N | C | |
| Seq ID NO 468 | 372 | L | L | E | T | T | I | G | N | S | |
| Seq ID NO 469 | 373 | L | L | E | T | T | I | W | D | C | nme-atrazine, |
| | | | | | | | | | | | amino- |
| | | | | | | | | | | | atrazine, |
| | | | | | | | | | | | atratone |
| Seq ID NO 470 | 374 | L | L | E | T | T | I | W | D | S | |
| Seq ID NO 471 | 375 | L | L | E | T | T | I | W | N | C | |
| Seq ID NO 472 | 376 | L | L | E | T | T | I | W | N | S | |
| Seq ID NO 473 | 377 | L | L | E | T | T | L | G | D | C | |
| | | | | | | | | | | | amino- |
| | | | | | | | | | | | atrazine, |
| | | | | | | | | | | | ametryn, |
| | | | | | | | | | | | atratone |
| Seq ID NO 474 | 378 | L | L | E | T | T | L | G | D | S | |
| Seq ID NO 475 | 379 | L | L | E | T | T | L | G | N | C | |
| Seq ID NO 476 | 380 | L | L | E | T | T | L | G | N | S | |
| Seq. ID NO 477 | 381 | L | L | E | T | T | L | W | D | C | nme-atrazine, |
| | | | | | | | | | | | amino- |
| | | | | | | | | | | | atrazine, |
| | | | | | | | | | | | ametryn, |
| | | | | | | | | | | | atratone |
| Seq ID NO 478 | 382 | L | L | E | T | T | L | W | D | S | ametryn |
| Seq ID NO 479 | 383 | L | L | E | T | T | L | W | N | C | ametryn |
| Seq ID NO 480 | 384 | L | L | E | I | T | L | W | N | C | |
| Seq ID NO 481 | 385 | L | V | D | I | P | I | G | D | C | |
| Seq ID NO 482 | 386 | L | V | D | I | P | I | G | D | S | |
| Seq ID NO. 483 | 387 | L | V | D | I | P | I | G | N | C | |
| Seq ID NO 484 | 388 | L | V | D | I | P | I | G | N | S | |
| Seq ID NO 485 | 389 | L | V | D | I | P | I | W | D | C | |
| Seq ID NO 486 | 390 | L | V | D | I | P | I | W | D | S | |
| Seq ID NO 487 | 391 | L | V | D | I | P | I | W | N | C | |
| Seq ID NO 488 | 392 | L | V | D | I | P | I | W | N | S | |
| Seq ID NO 489 | 393 | L | V | D | I | P | L | G | D | C | |
| Seq ID NO 490 | 394 | L | V | D | I | P | L | G | D | S | |
| Seq ID NO 491 | 395 | L | V | D | I | P | L | G | N | S | |
| Seq ID NO 492 | 396 | L | V | D | I | P | L | W | D | C | |
| Seq ID NO 493 | 397 | L | V | D | I | P | L | W | D | C | |
| Seq ID NO 494 | 398 | L | V | D | I | P | L | W | D | S | |
| Seq ID NO 495 | 399 | L | V | D | I | P | L | W | N | C | |
| Seq ID NO 496 | 400 | L | V | D | I | P | L | W | N | S | |
| Seq ID NO 497 | 401 | L | V | D | I | T | I | G | D | C | |
| Seq ID NO 498 | 402 | L | V | D | I | T | I | G | D | S | |
| Seq ID NO 499 | 403 | L | V | D | I | T | I | G | N | C | |
| Seq ID NO 500 | 404 | L | V | D | I | T | I | G | N | S | |
| Seq ID NO 501 | 405 | L | V | D | I | T | I | W | D | C | |
| Seq ID NO 502 | 406 | L | V | D | I | T | I | W | D | S | |
| Seq ID NO 503 | 407 | L | V | D | I | T | I | W | N | C | |
| Seq ID NO 504 | 408 | L | V | D | I | T | I | W | N | S | |
| Seq ID NO. 505 | 409 | L | V | D | I | T | L | G | D | C | |
| Seq ID NO 506 | 410 | L | V | D | I | T | L | G | D | S | |
| Seq ID NO 507 | 411 | L | V | D | I | T | L | G | N | C | |
| Seq ID NO 508 | 412 | L | V | D | I | T | L | G | N | S | |
| Seq ID NO 509 | 413 | L | V | D | I | T | L | W | D | C | |
| Seq ID NO 510 | 414 | L | V | D | I | T | L | W | D | S | |
| Seq ID NO 511 | 415 | L | V | D | I | T | L | W | N | C | |
| Seq ID NO 512 | 416 | L | V | D | I | T | L | W | N | S | |
| Seq ID NO 513 | 417 | L | V | D | T | P | I | G | 0 | C | |
| Seq ID NO 514 | 418 | L | V | D | T | P | I | G | 0 | S | |
| Seq ID NO 515 | 419 | L | V | D | T | P | I | G | N | C | |
| Seq ID NO 516 | 420 | L | V | D | T | P | I | G | N | S | |
| Seq ID NO 517 | 421 | L | V | D | T | P | I | W | D | C | |
| Seq ID NO 518 | 422 | L | V | D | T | P | I | W | D | S | |
| Seq ID NO 519 | 423 | L | V | D | T | P | I | W | N | C | |
| Seq ID NO 520 | 424 | L | V | D | T | P | I | W | N | S | |
| Seq ID NO 521 | 425 | L | V | D | T | P | L | G | D | C | |
| Seq ID NO 522 | 426 | L | V | D | T | P | L | G | D | S | |
| Seq ID NO 523 | 427 | L | V | D | T | P | L | G | N | C | |
| Seq ID NO 524 | 428 | L | V | D | T | P | L | G | N | S | |
| Seq ID NO 525 | 429 | L | V | D | T | P | L | W | D | C | |
| Seq ID NO 526 | 430 | L | V | D | T | P | L | W | D | S | |
| Seq ID NO 527 | 431 | L | V | D | T | P | L | W | N | C | |
| Seq ID NO 528 | 432 | L | V | D | T | P | L | W | N | S | |
| Seq ID NO 529 | 433 | L | V | D | T | T | I | G | D | C | |
| Seq ID NO 530 | 434 | L | V | D | T | T | I | G | D | S | |
| Seq ID NO 531 | 435 | L | V | D | T | T | I | G | N | C | |
| Seq ID NO 532 | 436 | L | V | D | T | T | I | G | N | S | |
| Seq ID NO 533 | 437 | L | V | D | T | T | I | W | D | C | |
| Seq ID NO 534 | 438 | L | V | D | T | T | I | W | D | S | |
| Seq ID NO 535 | 439 | L | V | D | T | T | I | W | N | C | |
| Seq ID NO 536 | 440 | L | V | D | T | T | I | W | N | S | |
| Seq ID NO 537 | 441 | L | V | D | T | T | L | G | D | C | amino- |
| | | | | | | | | | | | propazine |
| Seq ID NO 538 | 442 | L | V | D | T | T | L | G | D | S | |
| Seq ID NO 539 | 443 | L | V | D | T | T | L | G | N | C | |
| Seq ID NO 540 | 444 | L | V | D | T | T | L | G | N | S | |
| Seq ID NO 541 | 445 | L | V | D | T | T | L | W | D | C | |
| Seq ID NO 542 | 446 | L | V | D | T | T | L | W | D | S | |
| Seq ID NO 543 | 447 | L | V | D | T | T | L | W | N | C | |
| Seq ID NO 544 | 448 | L | V | D | T | T | L | W | N | S | |
| Seq ID NO 545 | 449 | L | V | E | I | P | I | G | D | C | |
| Seq ID NO 546 | 450 | L | V | E | I | P | I | G | D | S | |
| Seq ID NO 547 | 451 | L | V | E | I | P | I | G | N | C | |
| Seq ID NO 548 | 452 | L | V | E | I | P | I | G | N | S | |
| Seq ID NO 549 | 453 | L | V | E | I | P | I | W | D | C | |
| Seq ID NO 550 | 454 | L | V | E | I | P | I | W | D | S | |
| Seq ID NO 551 | 455 | L | V | E | I | P | I | W | N | C | |
| Seq ID NO 552 | 456 | L | V | E | I | P | I | W | N | S | |
| Seq ID NO 553 | 457 | L | V | E | I | P | L | G | D | C | |
| Seq ID NO 554 | 458 | L | V | E | I | P | L | G | D | S | |
| Seq ID NO 555 | 459 | L | V | E | I | P | L | G | N | C | |
| Seq ID NO 556 | 460 | L | V | E | I | P | L | G | N | S | |
| Seq ID NO 557 | 461 | L | V | E | I | P | L | W | D | C | |
| Seq ID NO. 558 | 462 | L | V | E | I | P | L | W | D | S | |
| Seq ID NO 559 | 463 | L | V | E | I | P | L | W | N | C | |
| Seq ID NO 560 | 464 | L | V | E | I | F | L | W | N | S | |
| Seq ID NO 561 | 465 | L | V | E | I | T | I | G | D | C | NME- |
| | | | | | | | | | | | propazine |
| Seq ID NO 562 | 466 | L | V | E | I | T | I | G | D | S | prometryn |
| Seq ID NO 563 | 467 | L | V | E | I | T | I | G | N | C | |
| Seq ID NO 564 | 468 | L | V | E | I | T | I | G | N | S | |
| Seq ID NO 565 | 469 | L | V | E | I | T | I | W | D | C | NME- |
| | | | | | | | | | | | propazine |
| Seq ID NO 566 | 470 | L | V | E | I | T | I | W | D | S | |
| Seq ID NO 567 | 471 | L | V | E | I | T | I | W | N | C | |
| Seq ID NO 568 | 472 | L | V | E | I | T | I | W | N | S | |
| Seq ID NO 569 | 473 | L | V | E | I | T | L | G | D | C | NME- |
| | | | | | | | | | | | propazine |
| Seq ID NO 570 | 474 | L | V | E | I | T | L | G | D | S | |
| Seq ID NO 571 | 475 | L | V | E | I | T | L | G | N | C | |
| Seq ID NO 572 | 476 | L | V | E | I | T | L | G | N | S | |
| Seq ID NO 573 | 477 | L | V | E | I | T | L | W | D | C | NME- |
| | | | | | | | | | | | propazine |
| Seq ID NO 574 | 478 | L | V | E | I | T | L | W | D | S | |
| Seq ID NO 575 | 479 | L | V | E | I | T | L | W | N | C | |
| Seq ID NO 576 | 480 | L | V | E | I | T | L | W | N | S | |
| Seq ID NO 577 | 481 | L | V | E | T | P | I | G | D | C | |
| Seq ID NO 578 | 482 | L | V | E | T | P | I | G | D | S | prometryn |
| Seq ID NO 579 | 483 | L | V | E | T | P | I | G | N | C | |
| Seq ID NO 580 | 484 | L | V | E | T | P | I | G | N | S | |
| Seq ID NO 581 | 485 | L | V | E | T | P | I | W | D | C | |
| Seq ID NO 582 | 486 | L | V | E | T | P | I | W | C | S | |
| Seq. ID NO 583 | 487 | L | V | E | T | P | I | W | N | C | |
| Seq ID NO 584 | 488 | L | V | E | T | P | I | W | N | S | |
| Seq ID NO 585 | 489 | L | V | E | T | P | L | G | D | C | |
| Seq ID NO 586 | 490 | L | V | E | T | P | L | G | D | S | |
| Seq ID NO 587 | 491 | L | V | E | T | P | L | G | N | C | |
| Seq ID NO 588 | 492 | L | V | E | T | P | L | G | N | S | |
| Seq ID NO 589 | 493 | L | V | E | T | P | L | W | D | C | |
| Seq ID NO 590 | 494 | L | V | E | T | P | L | W | C | S | |
| Seq ID NO 591 | 495 | L | V | E | T | P | L | W | N | C | |
| Seq ID NO 592 | 496 | L | V | E | T | P | L | W | N | S | |
| Seq ID NO 593 | 497 | L | V | E | T | T | I | G | D | C | prometryn, |
| | | | | | | | | | | | amino- |
| Seq ID NO 594 | 498 | L | V | E | T | T | I | D | D | S | prometryn |
| Seq ID NO 595 | 499 | L | V | E | T | T | I | G | N | C | |
| Seq ID NO 596 | 500 | L | V | E | T | T | I | G | N | S | |
| Seq ID NO 597 | 501 | L | V | E | T | T | I | W | D | C | prometryn, |
| | | | | | | | | | | | NME- |
| Seq ID NO 598 | 502 | L | V | E | T | T | I | W | D | S | prometryn |
| Seq ID NO 599 | 503 | L | V | E | T | T | I | W | N | C | |
| Seq ID NO 600 | 504 | L | V | E | T | T | I | W | N | S | propazine |
| Seq. ID NO 601 | 505 | L | V | E | T | T | L | G | D | C | nme-atrazine, |
| | | | | | | | | | | | prometryn, |
| | | | | | | | | | | | amino- |
| Seq ID NO 602 | 506 | L | V | E | T | T | L | G | D | S | prometryn, |
| | | | | | | | | | | | amino- |
| | | | | | | | | | | | propazine, |
| Seq ID NO. 603 | 507 | L | V | E | T | T | L | G | N | C | amino- |
| | | | | | | | | | | | propazine |
| Seq ID NO 604 | 508 | L | V | E | T | T | L | G | N | S | propazine |
| Seq ID NO 605 | 509 | L | V | E | T | T | L | W | D | C | nme-atrazine, |
| | | | | | | | | | | | prometryn, |
| | | | | | | | | | | | amino- |
| | | | | | | | | | | | propazine, |
| Seq ID NO 606 | 510 | L | V | E | T | T | L | W | D | S | prometryn, |
| | | | | | | | | | | | NME- |
| | | | | | | | | | | | propazine |
| Seq ID NO 607 | 511 | L | V | E | T | T | L | W | N | C | |
| Seq ID NO 608 | 512 | L | V | E | T | T | L | W | N | S | propazine |

What is claimed is:

1. An isolated or recombinant nucleic acid, comprising:
a polynucleotide sequence selected from the group consisting of:
(a) SEQ ID NO: 1 to SEQ ID NO: 48 or a complementary polynucleotide sequence thereof;
(b) a polynucleotide sequence encoding a polypeptide selected from SEQ ID NO: 49 to SEQ ID NO: 608, or a complementary polynucleotide sequence thereof;
(c) a polynucleotide sequence which hybridizes under highly stringent conditions over substantially the entire length of polynucleotide sequence (a) or (b); and,
(d) a polynucleotide sequence comprising a fragment of (a), (b), or (c), which fragment encodes a polypeptide having triazine hydrolase activity, which fragment is unique as compared to a nucleic acid corresponding to U55933 or AF312304.

2. The nucleic acid of claim 1, which nucleic acid comprises a polynucleotide that encodes a hydrolase.

3. The nucleic acid of 2, which hydrolase hydrolyzes one or more of aminoatrazine, atrazine, triazine, atratone, N-methylatrazine, ametryn, aminopropazine, propazine, prometon, N-methylpropazine, prometryn, aminomorphazine, morphazine, morphatryn, morphaton, or N-methylmorphazine.

4. An isolated or recombinant nucleic acid comprising a polynucleotide sequence encoding a polypeptide, the polypeptide comprising:
an amino acid sequence comprising at least 20 contiguous amino acids of any one of SEQ ID NO: 49-608, which amino acid sequence is unique as compared to a polypeptide encoded by nucleic acids U55933 and AF312304.

5. The nucleic acid of claim 4, wherein the encoded polypeptide is about 450 to about 500 amino acids in length or about 474 amino acids in length.

6. The nucleic acid of claim 4, wherein the encoded polypeptide has triazine hydrolase activity.

7. The nucleic acid of claim 4, wherein the encoded polypeptide comprises at least 50 contiguous amino acid residues of any one of SEQ ID NO: 49-608.

8. The nucleic acid of claim 4, wherein the encoded polypeptide comprises at least 100 contiguous amino acid residues of any one of SEQ ID NO: 49-608.

9. The nucleic acid of claim 4, wherein the encoded polypeptide comprises at least 150 contiguous amino acid residues of any one of SEQ ID NO: 49-608.

10. The nucleic acid of claim 4, wherein the encoded polypeptide comprises an amino acid sequence selected from the group consisting of: SEQ ID NO: 49-608.

11. The nucleic acid of claim 4, comprising a polynucleotide sequence selected from the group consisting of: SEQ ID NO: 1-48.

12. A cell comprising the nucleic acid of claim 1 or 4.

13. The cell of claim 12, wherein the cell expresses a polypeptide encoded by the nucleic acid.

14. A vector comprising the nucleic acid of claim 1 or 4.

15. The vector of claim 14, wherein the vector comprises a plasmid, a cosmid, a phage, or a virus.

16. The vector of claim 14, wherein the vector is an expression vector.

17. A cell transduced by the vector of claim 14.

18. A remediation composition comprising a cell comprising the polypeptide of claim 1 or 4.

19. The remediation composition of claim 18, wherein the remediation composition is suitable for treating soil or water.

20. A remediation composition comprising the polypeptide of claim 1 or 4.

21. A composition produced by digesting one or more nucleic acid of claim 1 or 4 with a restriction endonuclease, an RNAse, or a DNAse.

22. A composition produced by a process comprising incubating one or more nucleic acid of claim 1 or 4 in the presence of deoxyribonucleotide triphosphates and a nucleic acid polymerase.

23. The composition of claim 22, wherein the nucleic acid polymerase is a thermostable polymerase.

24. A composition comprising one or more polypeptide from claims 1 or 4.

25. The composition of claim 24, wherein the composition comprises a library comprising at least ten nucleic acids.

26. An isolated or recombinant polypeptide encoded by the nucleic acid of acid claim 1 or 4.

27. The isolated or recombinant polypeptide of claim 26, comprising a sequence selected from the group consisting of: SEQ ID NO: 49-608.

28. The polypeptide of claim 26, having triazine hydrolase activity of at least 50,000 nM per hour.

29. The polypeptide of claim 26, having triazine hydrolase activity of at least about 2-fold to at least about 200-fold greater than an atrazine chlorohydrolase corresponding to U55933.

30. A polypeptide comprising at least 100 contiguous amino acids of a protein encoded by a polynucleotide sequence, the polynucleotide sequence selected from the group consisting of:
(a) SEQ ID NO: 1 to SEQ ID NO: 48;
(b) a coding polynucleotide sequence that encodes a first polypeptide selected from SEQ ID NO: 49-608; and,
(c) a complementary polynucleotide sequence which hybridizes under highly stringent conditions over substantially an entire length of a polynucleotide sequence of (a) or (b).

31. The polypeptide of claim 30, which polypeptide comprises triazine hydrolase activity.

32. The polypeptide of claim 30, comprising at least 150 contiguous amino acids of the encoded protein.

33. The polypeptide of claim 30, comprising at least 250 contiguous amino acids of the encoded protein.

34. The polypeptide of claim 30, comprising an amino acid sequence selected from the group consisting of: SEQ ID NO: 49-608.

35. The polypeptide of claim 30, further comprising a secretion/localization sequence.

36. The polypeptide of claim 30, further comprising a polypeptide purification subsequence.

37. The polypeptide of claim 36, wherein the sequence that facilitates purification is selected from the group consisting of: an epitope tag, a FLAG tag, a polyhistidine tag, and a GST fusion.

38. The polypeptide of claim 30, further comprising a Met at the N-terminus.

39. The polypeptide of claim 30, comprising a modified amino acid.

40. The polypeptide of claim 39, wherein the modified amino acid is selected from the group consisting of: a glycosylated amino acid, a PEGylated amino acid, a farnesylated amino acid, an acetylated amino acid, and a biotinylated amino acid.

41. A polypeptide which is specifically bound by a polyclonal antisera raised against one or more antigen, the antigen comprising the sequence of SEQ ID NO: 49-608, or a fragment thereof, wherein the antisera is subtracted with a naturally occurring hydrolase polypeptide corresponding to U55933 or a triazine hydrolase homologue nucleic acid that is present in a public database such as GenBank™ at the time of filing of the subject application.

42. An antibody or antisera produced by administering the polypeptide of claim 41 to a mammal, which antibody specifically binds one or more antigen, the antigen comprising a polypeptide comprising one or more of the amino acid sequences of SEQ ID NO: 49-608, which antibody does not specifically bind to a naturally occurring or recombinant hydrolase polypeptide corresponding to U55933.

43. An antibody or antisera which specifically binds a polypeptide, the polypeptide comprising a sequence selected from the group consisting of: SEQ ID NO: 49-608, wherein the antibody does not specifically bind to naturally hydrolase polypeptide corresponding to U55933.

44. A method of producing a polypeptide, the method comprising:
introducing into a population of cells a nucleic acid of claim 1 or 4, the nucleic acid operatively linked to a regulatory sequence effective to produce the encoded polypeptide;
culturing the cells in a culture medium to produce the polypeptide; and,
isolating the polypeptide from the cells or from the culture medium.

45. A method of producing a polypeptide, the method comprising
introducing into a population of cells a recombinant expression vector comprising the nucleic acid of claim 1 or 4;
culturing the cells in a culture medium to produce the polypeptide encoded by the expression vector; and,
isolating the polypeptide from the cells or from the culture medium.

46. A method of treating a sample comprising atrazine or a triazine derivative comprising:
adding a composition to a sample comprising atrazine or a triazine derivative,
wherein the composition comprises a polypeptide encoded by a nucleic acid of claim 1 or 4.

47. A method of DNA shuffling, the method comprising:
recursively recombining one or more nucleic acid of claim 1 or 4 with one or more additional nucleic acid, the additional nucleic acid encoding a triazine hydrolase homologue or subsequence thereof.

48. The method of claim 47, wherein said recursive recombination produces at least one library of recombinant triazine hydrolase nucleic acids.

49. A nucleic acid library produced by the method of claim 48.

50. A population of cells comprising the library of claim 49.

51. A recombinant hydrolase nucleic acid produced by the method of claim 48.

52. A cell comprising the nucleic acid of claim 51.

53. The method of claim 47, wherein the recursive recombination is performed in vitro.

54. The method of claim 47, wherein the recursive recombination is performed in vivo.

55. A method of producing a modified triazine hydrolase nucleic acid homologue comprising mutating a nucleic acid of claim 1 or 4.

56. The modified hydrolase nucleic acid homologue produced by the method of claim 55.

57. A computer or computer readable medium comprising a database comprising a sequence record comprising one or more character string corresponding to a nucleic acid or protein sequence selected from SEQ ID NO: 1 to SEQ ID NO: 608.

58. An integrated system comprising a computer or computer readable medium comprising a database comprising one or more sequence records, each comprising one or more character strings corresponding to a nucleic acid or protein sequence selected from SEQ ID NO: 1 to SEQ ID NO: 608, the integrated system further comprising a user input interface allowing a user to selectively view one or more sequence record.

59. The integrated system of claim 58, the computer or computer readable medium comprising an alignment instruction set which aligns the character strings with one or more additional character string corresponding to a nucleic acid or protein sequence.

60. The integrated system of claim 59, wherein the instruction set comprises one or more of: a local homology comparison determination, a homology alignment determination, a search for similarity determination, and a BLAST determination.

61. The integrated system of claim 59, further comprising a user readable output element which displays an alignment produced by the alignment instruction set.

62. The integrated system of claim 58, the computer or computer readable medium further comprising an instruction set which translates one or more nucleic acid sequence comprising a sequence selected from SEQ ID NO: 1 to SEQ ID NO: 48, into an amino acid sequence.

63. The integrated system of claim 58, the computer or computer readable medium further comprising an instruction set for reverse-translating one or more amino acid sequence comprising a sequence selected from SEQ ID NO: 49-608, into a nucleic acid sequence.

64. The integrated system of claim 63, wherein the instruction set selects the nucleic acid sequence by applying a codon usage instruction set or an instruction set which determines sequence identity to a test nucleic acid sequence.

65. A method of using a computer system to present information pertaining to at least one of a plurality of sequence records stored in a database, said sequence records each comprising one or more character string corresponding to SEQ ID NO: 1 to SEQ ID NO: 608, the method comprising:
determining a list of one or more character strings corresponding to one or more of SEQ ID NO: 1 to SEQ ID NO: 608 or a subsequence thereof;
determining which character strings of said list are selected by a user; and,
displaying the selected character strings, or aligning the selected character strings with an additional character string.

66. The method of claim 65, further comprising displaying an alignment of the selected character string with the additional character string.

67. The method of claim 65, further comprising displaying the list.

68. A nucleic acid which comprises a unique subsequence in a nucleic acid selected from SEQ ID NO: 1 to SEQ ID NO: 48, wherein the unique subsequence is unique as compared to a nucleic acid corresponding to U55933 or AF312304.

69. A polypeptide which comprises a unique subsequence in a polypeptide selected from: SEQ ID NO: 49-608, wherein the unique subsequence is unique as compared to a polypeptide corresponding to U55933 or AF312304.

70. A target nucleic acid which hybridizes under stringent conditions to a unique coding oligonucleotide which encodes a unique subsequence in a polypeptide selected from: SEQ ID NO: 49-608, wherein the unique subsequence is unique as compared to a polypeptide corresponding to U55933 or AF312304.

71. The nucleic acid of claim 70, wherein the stringent conditions are selected such that a perfectly complementary oligonucleotide to the coding oligonucleotide hybridizes to the coding oligonucleotide with at least a 5× higher signal to noise ratio than for hybridization of the perfectly complementary oligonucleotide to a control nucleic acid corresponding to U59933, wherein the target nucleic acid hybridizes to the unique coding oligonucleotide with at least a 2× higher signal to noise ratio as compared to hybridization of the control nucleic acid to the coding oligonucleotide.

72. A nucleic acid encoding a triazine hydrolase polypeptide, which triazine hydrolase polypeptide is at least about 70% identical to the polypeptide of SEQ ID NO: 49, wherein the polypeptide comprises a leucine or a phenylalanine at position 84, a leucine or an alanine at position 92, a glutamic acid at position 125, a threonine at position 217, a threonine at position 219, an isoleucine or leucine at position 253, a glycine or a tryptophan at position 255, an asparagine or an aspartic acid at position 328, and a cysteine or a serine at position 331 and wherein the polypeptide is unique as compared to the polypeptide encoded by nucleic acid U55933 or AF312304.

73. A polypeptide that is at least about 70% identical to the polypeptide of SEQ ID NO: 49, wherein the polypeptide comprises a leucine or a phenylalanine at position 84, a leucine or an alanine at position 92, a glutamic acid at position 125, a threonine at position 217, a threonine at position 219, an isoleucine or leucine at position 253, a glycine or a tryptophan at position 255, an asparagine or an aspartic acid at position 328, and a cysteine or a serine at position 331 and wherein the polypeptide is unique as compared to the polypeptide encoded by the nucleic acid U55933 or AF312304.

74. A polypeptide, which polypeptide is at least about 70% identical to SEQ ID NO: 49, and comprises a unique amino acid at positions 84, 92, 125, 217, 219, 253, 255, 328, and 331 as compared to the polypeptide encoded by nucleic acid U55933 or AF312304.

75. A nucleic acid encoding a polypeptide, which polypeptide is at least about 70% identical to SEQ ID NO: 49, and comprises a unique amino acid at positions 84, 92, 125, 217, 219, 253, 255, 328, and 331 as compared to the polypeptide encoded by nucleic acid U55933 or AF312304.

76. An isolated or recombinant polypeptide comprising a modified SEQ ID NO:49, which modified SEQ ID NO: 49 comprises one or more modification selected from: L₈₄, L₉₂, D₁₂₅, I₂₁₇, P₂₁₉, L₂₅₃, W₂₅₅, D₃₂₈, and C₃₃₁.

77. The polypeptide of claim 76, wherein the polypeptide comprises atrazine hydrolase activity.

78. The polypeptide of claim 77, wherein the modified SEQ ID NO:49 comprises a set of modifications selected from:
(a) L₉₂, E₁₂₅, T₂₁₇, P₂₁₉, L₂₅₃, W₂₅₅, and S₃₃₁;
(b) L₉₂, E₁₂₅, T₂₁₇, P₂₁₉, L₂₅₃, G₂₅₅, and S₃₃₁;
(c) V₉₂, E₁₂₅, T₂₁₇, P₂₁₉, L₂₅₃, W₂₅₅, and S₃₃₁;
(d) V₉₂, E₁₂₅, T₂₁₇, P₂₁₉, L₂₅₃, G₂₅₅, and S₃₃₁;
(e) L₉₂, E₁₂₅, T₂₁₇, P₂₁₉, I₂₅₃, W₂₅₅, and S₃₃₁;
(f) L₉₂, E₁₂₅, T₂₁₇, P₂₁₉, I₂₅₃, G₂₅₅, and S₃₃₁;
(g) V₉₂, E₁₂₅, I₂₁₇, P₂₁₉, L₂₅₃, W₂₅₅, and S₃₃₁;
(h) V₉₂, E₁₂₅, I₂₁₇, P₂₁₉, L₂₅₃, G₂₅₅, and S₃₃₁;
(i) L₉₂, D₁₂₅, T₂₁₇, P₂₁₉, L₂₅₃, G₂₅₅, and S₃₃₁;
(j) V₉₂, E₁₂₅, T₂₁₇, P₂₁₉, L₂₅₃, W₂₅₅, and C₃₃₁;
(k) V₉₂, E₁₂₅, T₂₁₇, P₂₁₉, L₂₅₃, G₂₅₅, and C₃₃₁;
(l) V₉₂, E₁₂₅, T₂₁₇, P₂₁₉, I₂₅₃, W₂₅₅, and S₃₃₁;
(m) L₉₂, E₁₂₅, T₂₁₇, T₂₁₉, L₂₅₃, W₂₅₅, and S₃₃₁;
(n) V₉₂, E₁₂₅, T₂₁₇, P₂₁₉, I₂₅₃, G₂₅₅, and S₃₃₁;
(o) L₉₂, E₁₂₅, T₂₁₇, T₂₁₉, L₂₅₃, G₂₅₅, and S₃₃₁;
(p) V₉₂, D₁₂₅, T₂₁₇, P₂₁₉, L₂₅₃, W₂₅₅, and S₃₃₁;
(q) V₉₂, D₁₂₅, T₂₁₇, P₂₁₉, L₂₅₃, G₂₅₅, and S₃₃₁;
(r) V₉₂, E₁₂₅, T₂₁₇, T₂₁₉, L₂₅₃, W₂₅₅, and S₃₃₁; and,
(s) V₉₂, E₁₂₅, T₂₁₇, T₂₁₉, L₂₅₃, G₂₅₅, and S₃₃₁.

79. The polypeptide of claim 78, comprising a sequence selected from from: SEQ ID NO: 97: to SEQ ID NO: 608.

80. The polypeptide of claim 76, comprising atratone activity.

81. The polypeptide of claim 80, comprising a set of modifications selected from:
(a) F₈₄, L₉₂, E₁₂₅, L₂₅₃, G₂₅₅, D₃₂₈, and C₃₃₁;
(b) F₈₄, L₉₂, E₁₂₅, I₂₅₃, G₂₅₅, D₃₂₈, and C₃₃₁;
(c) F₈₄, L₉₂, E₁₂₅, L₂₅₃, W₂₅₅, D₃₂₈, and C₃₃₁;
(d) F₈₄, L₉₂, E₁₂₅, I₂₅₃, W₂₅₅, D₃₂₈, and C₃₃₁;
(e) L₈₄, L₉₂, E₁₂₅, L₂₅₃, G₂₅₅, D₃₂₈, and C₃₃₁;
(f) L₈₄, L₉₂, E₁₂₅, I₂₅₃, G₂₅₅, D₃₂₈, and C₃₃₁;
(g) F₈₄, L₉₂, D₁₂₅, L₂₅₃, G₂₅₅, D₃₂₈, and C₃₃₁;
(h) F₈₄, L₉₂, D₁₂₅, I₂₅₃, G₂₅₅, D₃₂₈, and C₃₃₁;
(i) L₈₄, L₉₂, E₁₂₅, L₂₅₃, W₂₅₅, D₃₂₈, and C₃₃₁;
(j) L₈₄, L₉₂, E₁₂₅, I₂₅₃, W₂₅₅, D₃₂₈, and C₃₃₁;
(k) F₈₄, L₉₂, D₁₂₅, L₂₅₃, W₂₅₅, D₃₂₈, and C₃₃₁;
(l) F₈₄, L₉₂, D₁₂₅, I₂₅₃, W₂₅₅, D₃₂₈, and C₃₃₁;
(m) L₈₄, L₉₂, D₁₂₅, L₂₅₃, G₂₅₅, D₃₂₈, and C₃₃₁;
(n) L₈₄, L₉₂, D₁₂₅, I₂₅₃, G₂₅₅, D₃₂₈, and C₃₃₁;
(o) L₈₄, L₉₂, D₁₂₅, L₂₅₃, W₂₅₅, D₃₂₈, and C₃₃₁; and,
(p) L₈₄, L₉₂, D₁₂₅, I₂₅₃, W₂₅₅, D₃₂₈, and C₃₃₁.

82. The polypeptide of claim 81, comprising a sequence selected from from: SEQ ID NO: 97: to SEQ ID NO: 608.

83. A polypeptide, which polypeptide is at least about 70% identical to SEQ ID NO: 49, and comprises a unique amino acid at positions 84 and 92, as compared to the polypeptide encoded by nucleic acid U55933 or AF312304.

84. A nucleic acid encoding a polypeptide, which polypeptide is at least about 70% identical to SEQ ID NO: 49, and comprises a unique amino acid at positions 84 and 92 as compared to nucleic acid U55933 or AF312304.

85. A polypeptide, which polypeptide is at least about 70% identical to the polypeptide encoded by nucleic acid U55933 or AF312304, and comprises a unique amino acid at positions 84 and 92, as compared to the polypeptide encoded by nucleic acid U55933 or AF312304.

86. A nucleic acid encoding a polypeptide, which polypeptide is at least about 70% identical to the polypeptide encoded by nucleic acid U55933 or AF312304 and which polypeptide comprises a unique amino acid at positions 84 and 92, as compared to the polypeptide encoded by nucleic acid U55933 or AF312304.

87. The polypeptide of claim 83 or claim 85, further comprising an asparagine at position 328 and a serine at position 331

88. The nucleic acid of claim 84 or claim 86, wherein the polypeptide further comprises an asparagine at position 328 and a serine at position 331
